(19) United States
(12) Patent Application Publication
(10) Pub. No.: US 2023/0414695 A1
(43) Pub. Date: Dec. 28, 2023

(54) USE OF SUGAR CANE EXTRACTS IN THE TREATMENT OR PREVENTION OF MICROBIAL INFECTIONS AND DYSBIOSIS

(71) Applicant: POLY GAIN PTE LTD, Singapore (SG)

(72) Inventors: Shane MITCHELL, Keysborough (AU); Barry KITCHEN, Keysborough (AU); Gregor MACNAB, Keysborough (AU); Matthew FLAVEL, Keysborough (AU)

(21) Appl. No.: 18/253,873

(22) PCT Filed: Nov. 23, 2021
(86) PCT No.: PCT/AU2021/051395

§ 371 Date: May 22, 2023

(30) Foreign Application Priority Data
Nov. 23, 2020 (AU) 2020904323

(51) Int. Cl. A61K 36/899; A61P 31/16; A61K 31/353; A61K 47/22

(52) U.S. Cl. CPC A61K 36/899; A61P 31/16; A61K 31/353; A61K 47/22

(57) ABSTRACT

Disclosed are methods and compositions comprising from about 0.00001 wt % to about 100 wt % of an extract derived from sugar cane for use in a variety of treatments, preventatives and control measures involving administration of an extract comprising from about 10 catechin equivalent (CE) g/L to about 50 CE g/L of polyphenols or from about 100 CE mg/g to about 500 CE mg/g of polyphenols to a subject. These treatments and preventative measures include antimicrobial activity, especially against respiratory viruses in humans as well as treatments for microsporidial infections, such as EHP in shrimps and prawns. Control measures include the reduction of methane generation in ruminants. Use of these compositions, for example in dairy cows, improves the health of the cows by minimizing mastitis whilst enhancing milk yield.

### TECHNICAL FIELD

The present disclosure is in the field of the inhibition or inactivation of viruses as well as the prevention or treatment of microbial infections in animals. These extracts are particularly useful against viruses that cause respiratory disease in animals.

The present disclosure also relates to the use of extracts derived from sugar cane comprising polyphenols for the inhibition or inactivation of microsporidian parasites as well as the prevention or treatment of microsporidian parasitic infections in animals. These extracts are particularly useful against Enterocytozoon hepatopenaei (EHP), a disease causing parasite in aquatic animals such as prawns and shrimp.

The present disclosure still further relates to the use of extracts derived from sugar cane comprising polyphenols for the inhibition or inactivation of protozoa as well as the prevention or treatment of protozoan infections in animals. These extracts are particularly useful against Coccidia and plasmodium species, the cause of malaria.

The present disclosure still further relates to the use of extracts derived from sugar cane comprising polyphenols for the inhibition or inactivation of Archae as well as the prevention or treatment of archaen infections in animals. These extracts are particularly useful for the treatment of dysbiosis and against methanogens that are present in ruminants.

The present disclosure still further relates to the use of extracts derived from sugar cane comprising polyphenols for the prevention or treatment of mastitis in ruminants. These extracts are particularly useful for the prevention or treatment of mastitis in milking cows.

The present disclosure still further relates to the use of extracts derived from sugar cane comprising polyphenols for the inhibition or inactivation of fungi as well as the prevention or treatment of fungal infections in animals, particularly against Ascomycota and Basidiomycota.

Mechanistically, the present disclosure relates to the use of extracts derived from sugar cane comprising polyphenols for the treatment, prevention or minimization of the cytokine storm in animals as is typically found in all pathogen invasions of animals.

Mechanistically, the present disclosure further relates to the use of extracts derived from sugar cane comprising polyphenols for the treatment, prevention or minimization of at least one of inflammation, tissue and organ damage in animals, arising from an acquired immune response, by modulation of the toll like receptors.

The present disclosure still further relates to the use of extracts derived from sugar cane comprising polyphenols as adjuvants either alone or in combination with known adjuvants, such as Freund's adjuvant.

### BACKGROUND

Respiratory disease epidemics have affected human populations throughout the globe in recent history. Severe Acute Respiratory Syndrome (SARS) emerged in China in 2002 and caused 8000 cases with a 10% mortality rate. Middle Eastern Respiratory Syndrome (MERS) MERS emerged in 2012 and caused 1700 cases with almost 40% mortality. Coronaviruses were identified as the infectious agents responsible for the SARS and MERS epidemics. These viruses are genetically diverse and have the ability to move in and out of human and new zoonotic hosts making them a challenge to counteract.

In December 2019, there was an outbreak of acute respiratory disease in Wuhan city, Hubei province, China with patients suffering from unexplainable pneumonia. By 7 Jan. 2020 it was confirmed that this new acute respiratory disease was caused by a novel coronavirus, SARS-CoV-2. Cases of the new disease, COVID-19, quickly throughout China and the world aided by virtue of international travel. On 11 Mar. 2020 The World Health Organisation (WHO) declared the disease as a pandemic.

Patients infected with SARS-CoV-2 may be asymptomatic. However, patients with symptoms may experience fever, dry cough and shortness of breath resulting from infection in the upper respiratory tract. Patients may also display flu-like symptoms. Where the infection is more severe and spread deeply into the lungs more serious illness and difficulty in breathing may develop. Secondary infection and/or non-pulmonary conditions (heart, renal complications) may also develop. Serious illness typically features pneumonia leading to acute respiratory disease syndrome (ARDS), which is one of the major causes of deaths from COVID-19.

There is no established treatment option for ARDS caused by SARS-CoV-2. Instead, supporting care and non-specific treatment protocols have been used to ameliorate patient's symptoms. Lung ventilator strategies either alone or in combination with the administration of, for example, broad spectrum antiviral or antibacterial agents or convalescent plasma remain the mainstay of available options in the absence of effective pharmaceutical therapy.

Further, candidates for pharmaceutical therapy of ARDS may lack the safety and the activity required. For example, steroids should not be generally used as they are known to decrease the immune response and may increase viral shedding. Attempts to treat patients in the prior SARS or MERS epidemics with steroids were not effective as were attempts with approved antivirals (ribavirin, lopinavir-ritonavir) and immunomodulators. Side effects can be observed with the above agents. For example anemia in the case of ribivarin.

More recently dexamethasone, a corticosteroid used in a wide range of conditions for its anti-inflammatory and immunosuppressant effects, has been tested in hospitalized patients with COVID-19 in the United Kingdom's national clinical trial RECOVERY and found to have benefits for critically ill patients. According to preliminary findings shared with WHO, for patients on ventilators, the treatment was shown to reduce mortality by about one third, and for patients requiring only oxygen, mortality was cut by about one fifth.

Although no specific antiviral has been approved, remdesivir has been found to reduce the time to recovery.

Whilst developing effective treatments is desirable, it is preferable that viral infection is prevented. Alternatively that it becomes possible to vaccinate against infection.

Prevention of corona virus infections is generally accomplished by physical means and disinfection. This includes effective handwashing, the use of face masks and shields, gloves and other forms of personal protective equipment. Simple handwashing with soap and water and hand sanitizers is effective to inactivate coronaviruses. Such measures prevent an individual from spreading virus from the hands to the oral or nasal cavities and thus infecting the respiratory system. Additionally, it prevents transmission through contacting other individuals.

Once an individual is infected by coronavirus in the respiratory system, the use of a mask acts to minimise the transmission of virus by the aerosol route. Similarly, a mask worn by an uninfected individual will reduce the probability of infection by inhaling viral containing aerosols.

However, handwashing products are not suitable for use in the oral or nasal cavities which is typically where initial infection occurs.

Thus there continues to exist a clear need for agents that are not only effective in handwashing but are safe and effective in use in the oral and nasal cavities.

More generally viral infections are the cause of serious disease in humans and animals. The development of suitable vaccines and effective treatments remain an important priority, especially where the underlying virus is highly infective.

Some viral families of clinical significance to humans and/or animals are as follows:

### Coronaviridae

SARS-CoV-1

SARS-CoV-2

MERS

Avian corona virus—responsible for significant economic losses in both broilers and layers.

Porcine epidemic diarrhea virus.

Bovine Corona virus—Causes a disease called winter dysentery, can cause severe diarrhea in calves and respiratory tract infections.

### Calciviridae

Norovirus—causes 200,000 deaths each year, no vaccine or specific treatment available. Responsible for ˜18% of all gastroenteritis cases.

Feline Calcivirus—Found in 50% of cats with upper respiratory infections.

### Orthomyxoviridae

Influenza A, B, C, D, all of which may infect humans.

Examples of Influenza A strains
H1N1 caused “Spanish flu” in 1918 and “Swine flu” in 2009.
H2N2 caused “Asian Flu”.
H3N2 caused “Hong Kong Flu”.
H5N1, “avian” or “bird flu”.]
H7N7 has unusual zoonotic potential.
H1N2 infects pigs and humans.

Salmon Isavirus—causes anaemia and losses on salmon farms

### Paramyxoviridae

Measles

Mumps

Hendra virus p Canine distemper virus—Kills many puppies, vaccine available but full protection from virus does not occur until 16 weeks of age.

Newcastle disease virus—Major disease in poultry, vaccine available but is an eye drop or spray.

### Pneumoviridae

Human metapneumovirus common symptoms include runny nose, congestion, sore throat, cough, headache, and fever, which can be seen as a cold. For people over 72 there is a risk that pneumonia will develop.

### Flaviviridae

West Nile Virus

Dengue Virus—390,000,000 infections per year

Tick borne encephalitis—No specific treatment. 10,000-12,000 cases per year

Zika—No treatment available, vaccine estimated in 2028

Hepatitis C

Theillers Disease Virus—Affects horses, common cause for hepatitis and liver failure in horses

Bovine viral diarrhea virus

Classical Swine Fever

Japanese encephalitis virus—68,000 cases per

Yellow Fever

### Tojaviridae

Several equine encephalitis viruses

Animal parasitism is responsible for a significant burden on animal welfare, which associated with economic costs spanning the agricultural industry. Three major groups of parasites are generally recognised including; protozoa, helminths and arthropods.

There are a diverse range of protozoan parasites, which are known to cause disease across both vertebrate and invertebrate species. These parasites are the causative agent for diseases including, but not limited to malaria; dysentery, giardia, cryptosporidium, Chaga's disease and coccidiosis.

Whilst some protozoa are specific to one host species, other protozoa are capable of infecting a range of host species. Therefore, the unchecked usage of anti-microbial drugs, particularly throughout the food supply chain is facing growing restrictions. This to ensure that these drugs are effective in emergency situations.

The requirement for non-antibiotic alternatives to help prevent disease developing is therefore an important means to improving global health and welfare outcomes, without destroying economic viability.

Microsporidian parasites are of economic significance in aqueous environments, particularly in prawn and shrimp aquaculture. Enterocytozoon hepatopenaei (EHP) is known to infect only the tubule epithelial cells of the hepatopancreas of prawns. Infection occurs by the extrusion or growth of the polar tubule of the microsporidia allowing it to attach to the hepatopancreas of prawns.

Some prawn species that are particularly susceptible to EHP are Penaeus monodon, Penaeus (Litopenaeus) vannamei and Penaeus (Litopenaeus) stylirostris.

An effective prevention or treatment means for infections, such as EHP, would be of considerable benefit in aquaculture industries.

Microbial agents are a threat to life on earth beyond the ability to cause disease in a wide variety of living organisms. This is because methanogenic Archaea are responsible for the largest contribution to agricultural greenhouse gas emissions.

Methanogens are not associated with a disease state or parasitism in animals from which they are isolated. However, the production of gases such as methane during fermentation has been identified as a factor involved in increased greenhouse gas emissions and global warming. Therefore methanogens are damaging the planet through their effect on the environment, rather than damaging individual animals through disease.

Ruminants produce the majority of agricultural methane, but are also vital for global food and textile supply. Therefore strategies are required that are able to mitigate the methane output, whilst maintaining or improving production yields.

Dietary strategies to modulate the microbiome of animals to inhibit the proliferation of methanogens, whilst promoting the communities of microbial species that support the health of the animal would meet these objectives.

Milk producing ruminants are subject to the development of mastitis. Mastitis is a bacterial infection that causes an inflammation of a cow's udder. The infection is caused by a wide spectrum of pathogens and, epidemiologically categorized into contagious and environmental mastitis. Mastitis is the most widespread and costly disease in the dairy cattle occurring throughout the world.

Electrical conductivity (EC) of milk has been introduced as an indicator trait for mastitis over the last decade. The EC is determined by the concentration of anions and cations. If the cow suffers from mastitis, the concentration of Na+ and Cl− in the milk increases, which leads to increased electrical conductivity of milk from the infected quarter. Most automatic milking systems have EC sensors incorporated for measuring EC during milking.

A description of the utility of the measurement of electrical conductivity is to be found in J Dairy Sci. 75, 606-614 (1992).

Electrical conductivity measurements in milk from healthy cows are generally between 4.0 and 5.0 milliSiemens (mS) at 25° C., while absolute electrical conductivity values for infected quarters usually range from 5.0 to 9.0 mS. In a typical EC profile for a cow infected with mastitis, the infected quarter shows a higher EC level during most of the milking, with spikes in the beginning and at the end of the milking.

A mild, uncomplicated mastitis in a single quarter is usually treated with intramammary antibiotics, such as β-lactams (amoxicillin, penicillin, and cephalosporins). Systemic antibiotics are used when more than one quarter is infected, when udder changes are marked or when the cow is obviously ill. Combination therapy, with both systemic and intramammary antibiotics, is believed to increase bacteriological cure rates and is used in severe cases of mastitis.

The main components of the economic impact of mastitis are related to the reduction in milk production, milk disposal, the cost of the medicines used in the treatment, the labour costs related to the treatment and the culling of animals. Together, these factors erode the milk income received by farmers. It is estimated that more than $150 million is lost to Australian dairy farmers each year through poor udder health. Moreover the use of antibiotics is now discouraged owing to the development of bacterial resistance and possible undesirable antibiotic residues in the meat and milk of animals for human consumption.

### SUMMARY

In one aspect of the disclosure there is provided a composition comprising from about 0.00001 wt % to about 100 wt % of an extract derived from sugar cane for use in the inhibition or inactivation of viruses or the prevention or treatment of viral infections in a subject, the extract comprising from about 10 catechin equivalent (CE) g/L to about 50 CE g/L of polyphenols or from about 100 CE mg/g to about 500 CE mg/g of polyphenols, and wherein the use is by a topical, a pulmonary, a respiratory, an intravenous or an oral route.

In another aspect of the disclosure there is provided a method of inhibiting or inactivating viruses or the prevention or treatment of viral infections in a subject comprising administering to the subject by a topical, a pulmonary, a respiratory, an intravenous or an oral route an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in the prevention or in the treatment of an aquatic animal by inhibition or inactivation of microsporidian parasites, the extract comprising from about 10 catechin equivalent (CE) g/L to about 50 CE g/L of polyphenols or from about 100 CE mg/g to about 500 CE mg/g of polyphenols, and wherein the use is by inclusion of the extract in an aquatic environment.

In another aspect of the disclosure there is provided a method of inhibiting or inactivating microsporidian parasites in an aquatic animal comprising administering to the animal via its aquatic environment an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in the prevention or in the treatment of protozoan infection in an animal by inhibition or inactivation of protozoa, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of inhibiting or inactivating protozoa in an animal comprising administering to the animal an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in the prevention or treatment of Archae infections in an animal by inhibition or inactivation of archaens, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of inhibiting or inactivating Archae in an animal comprising administering to the animal an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in the treatment of dysbiosis in an animal by inhibition or inactivation of methanogens, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of treating dysbiosis in an animal comprising administering to the animal an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in the prevention or in the treatment of fungal infection in an animal by inhibition or inactivation of fungi, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of inhibiting or inactivating fungi in an animal comprising administering to the animal an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of inhibiting or inactivating the lifecycle of protozoan parasites both in vertebrates and invertebrate animals or in an environment they inhabit, comprising administering to the animal, an effective amount of a composition of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in inhibiting or inactivating the lifecycle of protozoan parasites both in vertebrates and invertebrate animals or in an environment they inhabit, the extract comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of modulating the microbiome of animals, comprising administering to the animal, an effective amount of a composition of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in modulating the microbiome of animals, the extract comprising from about wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of mitigating greenhouse gas emissions by an animal by changing the composition of microbial communities associated with the animal by comprising administering to the animal, an effective amount of a composition of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in mitigating greenhouse gas emissions by an animal by changing the composition of microbial communities associated with the animal, the extract comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method for the prevention or treatment of mastitis in a ruminant animal comprising administering to the animal, an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in the prevention or treatment of mastitis in a ruminant animal, the extract comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of mitigating methane emissions by a ruminant by changing the composition of microbial communities associated with the ruminant and treating or preventing mastitis in the ruminant comprising administering to the ruminant, an effective amount of a composition of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in mitigating greenhouse gas emissions by an animal by changing the composition of microbial communities associated with the animal and treating or preventing mastitis in the ruminant, the extract comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of enhancing milk production in a ruminant comprising administering to the ruminant, an effective amount of a composition of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for, enhancing milk production in a ruminant, the extract comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of mitigating methane emissions by a ruminant by changing the composition of microbial communities associated with the ruminant whilst enhancing the growth of the ruminant comprising administering to the ruminant, an effective amount of a composition of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use in mitigating methane emissions by a ruminant by changing the composition of microbial communities associated with the ruminant whilst enhancing the growth of the ruminant, the extract comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

In another aspect of the disclosure there is provided a composition comprising from about 0.00001 wt % to about 100 wt % of an extract derived from sugar cane for use as an adjuvant either alone or in combination with a second adjuvant, the extract comprising from about 10 catechin equivalent (CE) g/L to about 50 CE g/L of polyphenols or from about 100 CE mg/g to about 500 CE mg/g of polyphenols.

In another aspect of the disclosure there is provided a method of improving the immune response of a vaccine comprising including a composition comprising from about 0.00001 wt % to about 100 wt % of an extract derived from sugar cane in a vaccine composition, the extract comprising from about 10 catechin equivalent (CE) g/L to about 50 CE g/L of polyphenols or from about 100 CE mg/g to about 500 CE mg/g of polyphenols.

### BRIEF DESCRIPTION OF DRAWINGS

FIG. 1 is a bar chart showing the results for Example 3 as described herein.

FIG. 2 is a bar chart showing the results for Example 5 as described herein.

FIG. 3 is a bar chart showing the results for Example 6 as described herein.

FIG. 4 is a bar chart showing the results for Example 7 as described herein.

FIG. 5 is a bar chart showing the results for Example 8 as described herein.

FIG. 6 is a bar chart showing the results for Example 14 as described herein.

FIG. 7 is a bar chart showing the results for Example 13 as described herein.

FIG. 8 is a graph representing the results achieved in Example 19 for a 0.5% composition.

FIG. 9 is a graph representing the results achieved in Example 19 for a 0.5% composition.

FIG. 10 are bar charts showing the results for Example 20 as described herein.

### DESCRIPTION OF EMBODIMENTS

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Unless specifically defined otherwise, all technical and scientific terms used herein shall be taken to have the same meaning as commonly understood by one of ordinary skill in the art (e.g., chemistry, biochemistry, cell culture, molecular biology and pharmacy). Further, unless otherwise required by context, singular terms shall include pluralities and plural terms shall include the singular. Thus, as used in this specification and the appended claims, the singular forms “a”, “an” and “the” include plural referents unless the context clearly indicates otherwise. Thus, the term “an subject” means “one or more subjects” unless the context clearly indicates otherwise.

“Administering” as used herein is to be construed broadly and includes administering an extract or a composition comprising the extract as described herein to a subject as well as providing an extract or composition comprising the extract as described herein to a cell.

The phrase “an effective amount” as used herein, refers to an amount which is sufficient to elicit the biological or medical response of a tissue, system, animal or human that is being sought by the researcher, veterinarian, medical doctor or other clinician. Undesirable effects, e.g. side effects, are sometimes manifested along with the desired effect; hence, a practitioner balances the potential benefits against the potential risks in determining what an appropriate “effective amount” is. The exact amount required varies from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using routine experimentation. By “ameliorate” is included relieving of adverse symptoms, inducing a state of comfort or wellbeing or removing or reducing biochemical, physiological or clinical markers of the disease or the condition.

The terms “treating”, “treat”, “treatment”, “improving”, “improve” or “improvement”, as used herein, include administering an effective amount of an extract of the present disclosure or a composition comprising the extract sufficient to reduce or delay the onset or progression of a specified condition, or to reduce or eliminate at least one symptom of the condition. As would be understood by those skilled in the art of treating a microbial infection, the term “treatment” includes that the infection is cured, however, it does not necessarily mean that the infection is completely cured.

The terms “preventing” or “prevent” as used herein, include administering an effective amount of an extract of the present disclosure or a composition comprising the extract sufficient to avoid the onset of a specified condition, or to avoid at least one symptom of the condition. As would be understood by those skilled in the art of preventing a condition, the term “preventing” includes that the condition is completely prevented, however, it does not necessarily mean that the condition is completely prevented.

“Subject” as used herein refers to an animal, such as mammal including a human who can benefit from the extracts derived from sugar cane, compositions containing the extracts and methods and uses described herein. There is no limitation on the type of animal that could benefit from the presently described extracts derived from sugar cane, compositions containing the extracts and methods and uses. A subject regardless of whether a human or non-human animal may be referred to as an individual, subject, animal, host or recipient as well as patient. of the present disclosure have applications in human medicine, human cosmetics, and veterinary medicine.

References to “compositions for use” are to be understood to encompass methods of this disclosure.

The term “about” as used herein refers to a range of +/−5% of the specified value.

The term “CE”, or “catechin equivalent” as used herein is a measure of total polyphenolic content, expressed as mg catechin equivalents/g crude material or g catechin equivalents/L crude material.

The term “sugar cane derived product” as used herein refers to products of the sugar cane milling and refining processes including, but not limited to, sugar, molasses, massecuite, bagasse, first expressed juice, mill mud, clarified sugar cane juice, clarified syrup, treacle, golden syrup, field trash, cane strippings, leaves, growing tips, pulp and dunder and combinations thereof. Dunder is the residue produced when a product such as sugar or molasses is fermented to give, for example, ethanol. Sugar cane dunder is also referred to as biodunder, stillage or vinasse. As used herein , the terms “dunder”, “bio-dunder”, “stillage” and “vinasse” are equivalent and used interchangeably.

Throughout this specification, various aspects and components of this disclosure can be presented in a range format. The range format is included for convenience and should not be interpreted as an inflexible limitation on the scope of this disclosure. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. In one embodiment, the extract derived from sugar cane of the present disclosure comprises at least about 10 CE g/L of polyphenols or at least about 150 mg CE/g of polyphenols. As explained above, the term “CE”, or “catechin equivalent” is a measure of total polyphenolic content, expressed as catechin equivalents mg/g extract derived from sugar cane or catechin equivalents g/L extract derived from sugar cane.

In one embodiment, the extract derived from sugar cane of the present disclosure comprises at least about 1, 2, 3, 4, 5, 6, 7, 8, 9, 10, 15, 20, 25, 30, 35, 40, 45, 50, 55, 60, 65, 70, 75, 80, 85, 90, 95, or 100 CE g/L of polyphenols.

In one embodiment, the extract derived from sugar cane of the present disclosure comprises at least about 10, 20, 30, 40, 50, 60, 70, 80, 90, 100, 110, 120, 130, 140, 150, 160, 170, 180, 190, 200, 210, 220, 230, 240, 250, 250, 275, 300, 325, 350, 375, 400, 425, 450, 500, 525, 550, 575, 600, 625, 650, 675, 700, 725, 750, 775 or 800 mg CE/g of polyphenols.

In one embodiment, the extract derived from sugar cane of the present disclosure comprises from about 1 CE g/L to about 50 CE g/L of polyphenols or from about 10 CE mg/g to about 500 CE mg/g of polyphenols.

In one embodiment, the extract derived from sugar cane of the present disclosure comprises from about 1 CE g/L to about 25 CE g/L of polyphenols or from about 10 CE mg/g to about 250 CE mg/g of polyphenols.

In one embodiment, the extract derived from sugar cane of the present disclosure comprises from about 1 CE g/L to about 10 CE g/L of polyphenols or from about 10 CE mg/g to about 100 CE mg/g of polyphenols.

In one embodiment, the extract derived from sugar cane of the present disclosure comprises from about 1 CE g/L to about 5 CE g/L of polyphenols or from about 10 CE mg/g to about 100 CE mg/g of polyphenols.

In one embodiment, the extract derived from sugar cane of the present disclosure comprises from about 5 CE g/L to about 50 CE g/L of polyphenols or from about 50 CE mg/g to about 500 CE mg/g of polyphenols.

In one embodiment, the extract derived from sugar cane of the present disclosure comprises from about 5 CE g/L to about 25 CE g/L of polyphenols or from about 50 CE mg/g to about 250 CE mg/g of polyphenols.

In one embodiment, the extract derived from sugar cane of the present disclosure comprises from about 5 CE g/L to about 10 CE g/L of polyphenols or from about 50 CE mg/g to about 100 CE mg/g of polyphenols.

In one embodiment, the extract derived from sugar cane of the present disclosure comprises from about 10 CE g/L to about 100 CE g/L of polyphenols or from about 100 CE mg/g to about 1000 CE mg/g of polyphenols.

In one embodiment, the extract derived from sugar cane of the present disclosure comprises from about 10 CE g/L to about 90 CE g/L of polyphenols or from about 100 CE mg/g to about 900 CE mg/g of polyphenols.

In one embodiment, the extract derived from sugar cane of the present disclosure comprises from about 10 CE g/L to about 80 CE g/L of polyphenols or from about 100 CE mg/g to about 800 CE mg/g of polyphenols.

In one embodiment, the extract derived from sugar cane of the present disclosure comprises from about 10 CE g/L to about 70 CE g/L of polyphenols or from about 100 CE mg/g to about 700 CE mg/g of polyphenols.

In one embodiment, the extract derived from sugar cane of the present disclosure comprises from about 10 CE g/L to about 60 CE g/L of polyphenols or from about 100 CE mg/g to about 600 CE mg/g of polyphenols.

In one embodiment, the extract derived from sugar cane of the present disclosure comprises from about 10 CE g/L to about 50 CE g/L of polyphenols or from about 100 CE mg/g to about 500 CE mg/g of polyphenols.

In one embodiment, the extract derived from sugar cane of the present disclosure comprises from about 10 CE g/L to about 25 CE g/L of polyphenols or from about 100 CE mg/g to about 250 CE mg/g of polyphenols.

In one embodiment, the extract derived from sugar cane of the present disclosure comprises from about 15 CE g/L to about 50 CE g/L of polyphenols or from about 150 CE mg/g to about 500 CE mg/g of polyphenols.

In one embodiment, the extract derived from sugar cane of the present disclosure comprises from about 15 CE g/L to about 25 CE g/L of polyphenols or from about 150 CE mg/g to about 250 CE mg/g of polyphenols.

In one embodiment, the extract derived from sugar cane of the present disclosure comprises from about 10 CE g/L to about 70 CE g/L of polyphenols or from about 100 CE mg/g to about 700 CE mg/g of polyphenols.

In one embodiment, the extract derived from sugar cane of the present disclosure comprises from about 10 CE g/L to about 60 CE g/L of polyphenols or from about 100 CE mg/g to about 600 CE mg/g of polyphenols.

In one embodiment, the extract derived from sugar cane of the present disclosure comprises from about 10 CE g/L to about 50 CE g/L of polyphenols or from about 100 CE mg/g to about 500 CE mg/g of polyphenols.

In one embodiment, the extract derived from sugar cane of the present disclosure comprises from about 15 CE g/L to about 40 CE g/L of polyphenols or from about 150 CE mg/g to about 400 CE mg/g of polyphenols.

In one embodiment, the extract derived from sugar cane of the present disclosure comprises from about 20 CE g/L to about 30 CE g/L of polyphenols or from about 200 CE mg/g to about 300 CE mg/g of polyphenols.

In one embodiment, the extract derived from sugar cane of the present disclosure comprises from about 20 CE g/L to about 27 g CE/L of polyphenols or from about 200 CE mg/g to about 270 CE mg/g of polyphenols.

In one embodiment, the extract derived from sugar cane of the present disclosure comprises from about 27 CE g/L to about 35 g CE/L of polyphenols or about 270 CE mg/g to about 350 CE mg/g of polyphenols.

In one embodiment, the extract derived from sugar cane of the present disclosure comprises from about 35 CE g/L to about 40 g CE/L of polyphenols or from about 350 CE mg/g to about 400 CE mg/g of polyphenols.

In one embodiment, the extract derived from sugar cane of the present disclosure comprises from about 40 CE g/L to about 50 g CE/L of polyphenols or from about 400 CE mg/g to about 500 CE mg/g of polyphenols.

In one embodiment, the extract derived from sugar cane of the present disclosure comprises from about 45 CE g/L to about 50 g CE/L of polyphenols or about 450 CE mg/g to about 500 CE mg/g of polyphenols.

The extract derived from sugar cane of the present disclosure may contain the flavonoid class of polyphenols. The extract derived from sugar cane may contain flavonoids in any amount. In one embodiment, the extract derived from sugar cane of the disclosure comprises at least about 1 CE g/L of flavonoids or at least about 10 CE mg/g of flavonoids.

In one embodiment, the extract derived from sugar cane of the present disclosure comprises from about 1 CE g/L to about 15 CE g/L of flavonoids or from about 10 CE mg/g to about 150 CE mg/g of flavonoids. In one embodiment, the extract derived from sugar cane of the present disclosure comprises from about 3 CE g/L to about 10 CE g/L of flavonoids or about 30 CE mg/g to about 100 CE mg/g of flavonoids. In one embodiment, the extract derived from sugar cane of the present disclosure comprises about 5 CE g/L to about 8 CE g/L of flavonoids or about 50 CE mg/g to about 80 CE mg/g of flavonoids. In one embodiment, the extract derived from sugar cane of the present disclosure comprises about 6 CE g/L to about 8 CE g/L of flavonoids or about 60 CE mg/g to about 80 CE mg/g of flavonoids. In one embodiment, the extract derived from sugar cane of the present disclosure comprises about 6.5 CE g/L to about 7.5 CE g/L of flavonoids or about 65 CE mg/g to about 75 CE mg/g of flavonoids.

The extract derived from sugar cane of the present disclosure may contain the proanthocyanidin class of polyphenols. The extract derived from sugar cane may contain proanthocyandins in any amount. In one embodiment, the extract derived from sugar cane of the present disclosure comprises at least about 1.5 CE g/L of proanthocyanidins or at least about 15 CE mg/g of proanthocyanidins. In one embodiment, the extract derived from sugar cane of the disclosure comprises at least about 1.8 CE g/L of proanthocyanidins or at least about 18 CE mg/g of proanthocyanidins. In one embodiment, the extract derived from sugar cane of the disclosure comprises about 1.5 CE g/L to about 2.5 CE g/L of proanthocyanidins or about 15 CE mg/g to about 25 CE mg/g of proanthocyanidins. In one embodiment, the extract derived from sugar cane of the disclosure comprises about 1.8 CE g/L to about 2.2 CE g/L of proanthocyanidins or about 18 CE mg/g to about 22 CE mg/g of proanthocyanidins.

The polyphenols of the extract derived from sugar cane of the present disclosure include, but are not limited to, one or more of syringic acid, chlorogenic acid, caffeic acid, vanillin, sinapic acid, p-coumaric acid, ferulic acid, gallic acid, vanillic acid, diosmin, diosmetin, apigenin, vitexin, orientin, homoorientin, swertisin, tricin, (+)-catechin, (−)-catechin gallate, (−) epicatechin, quercetin, kaempherol, myricetin, rutin, schaftoside, isoschaftoside, luteolin, scoparin and/or derivatives thereof. The polyphenols of the extract derived from sugar cane of the present disclosure may also include, but are not limited to, one or more of hydroxycinnamic acid, isoorientin, swertiajaponin, neocarlinoside, isovitexin, vicenin, and/or derivatives thereof.

The polyphenols of the extract derived from sugar cane also include conjugates, such as, for example, glycosides, glucosides, galactosides, galacturonides, ethers, esters, arabinosides, sulphates, phosphates, aldopentoses (xylose, arabinose) and aldohexoses.

In one embodiment, the extract derived from sugar cane of the present disclosure comprises syringic acid, chlorogenic acid, caffeic acid, vanillin, sinapic acid, diosmin, diosmetin, apigenin, vitexin, orientin, homoorientin, swertisin, and tricin and/or derivatives thereof.

In one embodiment, the extract derived from sugar cane of the present disclosure comprises syringic acid, chlorogenic acid and diosmin and/or derivatives thereof.

In one embodiment, the extract derived from sugar cane of the present disclosure comprises syringic acid. In one embodiment, the extract derived from sugar cane of the present disclosure comprises chlorogenic acid. In one embodiment, the extract derived from sugar cane of the present disclosure comprises diosmin. In one embodiment, the extract derived from sugar cane of the present disclosure comprises caffeic acid. In one embodiment, the extract derived from sugar cane of the present disclosure comprises vanillin. In one embodiment, the extract derived from sugar cane of the present disclosure comprises sinapic acid. In one embodiment, the extract derived from sugar cane of the present disclosure comprises vitexin. In one embodiment, the extract derived from sugar cane of the present disclosure comprises p-coumaric acid. In one embodiment, the extract derived from sugar cane of the present disclosure comprises ferulic acid. In one embodiment, the extract derived from sugar cane of the present disclosure comprises gallic acid. In one embodiment, the extract derived from sugar cane of the present disclosure comprises vanillic acid. In one embodiment, the extract derived from sugar cane of the present disclosure comprises diosmetin. In one embodiment, the extract derived from sugar cane of the present disclosure comprises apigenin. In one embodiment, the extract derived from sugar cane of the present disclosure comprises orientin. In one embodiment, the extract derived from sugar cane of the present disclosure comprises homoorientin. In one embodiment, the extract derived from sugar cane of the present disclosure comprises swertisin. In one embodiment, the extract derived from sugar cane of the present disclosure comprises tricin. In one embodiment, the extract derived from sugar cane of the present disclosure comprises (+)-catechin. In one embodiment, the extract derived from sugar cane of the present disclosure comprises (−)-catechin gallate. In one embodiment, the extract derived from sugar cane of the present disclosure comprises (−)-epicatechin. In one embodiment, the extract derived from sugar cane of the present disclosure comprises quercetin. In one embodiment, the extract derived from sugar cane of the present disclosure comprises kaempherol. In one embodiment, the extract derived from sugar cane of the present disclosure comprises myricetin. In one embodiment, the extract derived from sugar cane of the present disclosure comprises rutin. In one embodiment, the extract derived from sugar cane of the present disclosure comprises schaftoside. In one embodiment, the extract derived from sugar cane of the present disclosure comprises isoschaftoside. In one embodiment, the extract derived from sugar cane of the present disclosure comprises luteolin. In one embodiment, the extract derived from sugar cane of the present disclosure comprises scoparin. In one embodiment, the extract derived from sugar cane of the present disclosure comprises hydroxycinnamic acid. In one embodiment, the extract derived from sugar cane of the present disclosure comprises isoorientin. In one embodiment, the extract derived from sugar cane of the present disclosure comprises swertiajaponin. In one embodiment, the extract derived from sugar cane of the present disclosure comprises neocarlinoside. In one embodiment, the extract derived from sugar cane of the present disclosure comprises isovitexin. In one embodiment, the extract derived from sugar cane of the present disclosure comprises vicenin.

In one embodiment, syringic acid, chlorogenic acid and diosmin are the three most abundant polyphenols of the extract derived from sugar cane of the present disclosure.

In one embodiment, the extract derived from sugar cane of the disclosure comprises about 5-20 μg/g dry weight of syringic acid. In one embodiment, the extract derived from sugar cane of the disclosure comprises about 7-15 μg/g dry weight of syringic acid. In one embodiment, the extract derived from sugar cane of the disclosure comprises about 10-12 μg/g dry weight of syringic acid. In one embodiment, the extract derived from sugar cane of the disclosure, when present, comprises about 10.9 μg/g dry weight of syringic acid. The extract derived from sugar cane may be in a syrup form.

In one embodiment, the extract derived from sugar cane of the disclosure comprises about 50-200 μg/g dry weight of syringic acid. In one embodiment, the extract derived from sugar cane of the disclosure comprises about 90-130 μg/g dry weight of syringic acid. In one embodiment, the extract derived from sugar cane of the disclosure comprises about 100-120 μg/g dry weight of syringic acid. In one embodiment, the extract derived from sugar cane of the disclosure comprises about 107 μg/g dry weight of syringic acid. The extract derived from sugar cane may be in a powder form.

In one embodiment, the extract derived from sugar cane of the disclosure comprises about 1-15 μg/g dry weight of chlorogenic acid. In one embodiment, the extract derived from sugar cane of the disclosure comprises about 3-10 μg/g dry weight of chlorogenic acid. In one embodiment, the extract derived from sugar cane of the disclosure comprises about 5-8 μg/g dry weight of chlorogenic acid. In one embodiment, the extract derived from sugar cane of the disclosure comprises about 6.53 μg/g dry weight of chlorogenic acid. The extract derived from sugar cane may be in a syrup form.

In one embodiment, the extract derived from sugar cane of the disclosure comprises about 30-150 μg/g dry weight of chlorogenic acid. In one embodiment, the extract derived from sugar cane of the disclosure comprises about 60-90 μg/g dry weight of chlorogenic acid. In one embodiment, the extract derived from sugar cane of the disclosure comprises about 70-80 μg/g dry weight of chlorogenic acid. In one embodiment, the extract derived from sugar cane of the disclosure comprises about 74 μg/g dry weight of chlorogenic acid. The extract derived from sugar cane may be in a powder form.

In one embodiment, the extract derived from sugar cane of the disclosure comprises about 10-30 μg/g dry weight of diosmin. In one embodiment, the extract derived from sugar cane of the disclosure comprises about 15-25 μg/g dry weight of diosmin. In one embodiment, the extract derived from sugar cane of the disclosure comprises about 18-21 μg/g dry weight of diosmin. In one embodiment, the extract derived from sugar cane of the disclosure comprises about 19-45 μg/g dry weight of diosmin. The extract derived from sugar cane may be in a syrup form.

In one embodiment, the extract derived from sugar cane of the disclosure comprises about 100-300 μg/g dry weight of diosmin. In one embodiment, the extract derived from sugar cane of the disclosure comprises about 190-260 μg/g dry weight of diosmin. In one embodiment, the extract derived from sugar cane of the disclosure comprises about 210-240 μg/g dry weight of diosmin. In one embodiment, the extract derived from sugar cane of the disclosure comprises about 227 μg/g dry weight of diosmin. The extract derived from sugar cane may be in a powder form.

In one embodiment, the extract derived from sugar cane of the present disclosure comprises about 7-15 μg/g dry weight of syringic acid, and/or about 4-9 μg/g dry weight of chlorogenic acid, and/or about 0.1-0.5 μg/g dry weight of caffeic acid, about 0.05-0.3 μg/g dry weight of vanillin, and/or about 0.1-0.3 μg/g dry weight of sinapic acid, and/or about 15-25 μg/g dry weight of diosmin, and/or about 0.1-0.4 μg/g dry weight of orientin, and/or about 0.4-0.9 μg/g dry weight of swertisin, and/or about 0.05-0.3 μg/g dry weight of disomentin. The extract derived from sugar cane may be in a syrup form.

In one embodiment, the extract derived from sugar cane of the present disclosure comprises about 10-12 μg/g dry weight of syringic acid, and/or about 5-8 μg/g dry weight of chlorogenic acid, and/or about 0.2-0.4 μg/g dry weight of caffeic acid, and/or about 0.1-0.2 μg/g dry weight of vanillin, and/or about 0.1-0.25 μg/g dry weight of sinapic acid, and/or about 18-21 μg/g dry weight of diosmin, and/or about 0.2-0.3 μg/g dry weight of orientin, and/or about 0.5-0.8 μg/g dry weight of swertisin, and/or about 0.1-0.2 μg/g dry weight of disomentin. The extract derived from sugar cane may be in a syrup form.

In one embodiment, the extract derived from sugar cane of the present disclosure comprises about 10.9 μg/g dry weight of syringic acid, and/or about 6.53 μg/g dry weight of chlorogenic acid, and/or about 0.29 μg/g dry weight of caffeic acid, and/or about 0.153 μg/g dry weight of vanillin, and/or about 0.18 μg/g dry weight of sinapic acid, and/or about 19.45 μg/g dry weight of diosmin, and/or about 0.245 μg/g dry weight of orientin, and/or about 0.69 μg/g dry weight of swertisin, and/or about 0.15 μg/g dry weight of disomentin. The extract derived from sugar cane may be in a syrup form.

In one embodiment, the extract derived from sugar cane of the present disclosure comprises about 90-130 μg/g dry weight of syringic acid, and/or about 60-90 μg/g dry weight of chlorogenic acid, and/or about 4-10 μg/g dry weight of caffeic acid, and/or about 1-4 μg/g dry weight of vanillin, about 1-3 μg/g dry weight of sinapic acid, and/or about 190-260 μg/g dry weight of diosmin, and/or about 3-7 μg/g dry weight of orientin, and/or 3-8 μg/g dry weight of swertisin, and/or about 0.05-0.3 μg/g dry weight of disomentin. The extract derived from sugar cane may be in a powder form.

In one embodiment, the extract derived from sugar cane of the present disclosure comprises about 100-120 μg/g dry weight of syringic acid, and/or about 70-80 μg/g dry weight of chlorogenic acid, and/or about 6 - 8 μg/g dry weight of caffeic acid, about 2-3 μg/g dry weight of vanillin, and/or about 1.5-2.5 μg/g dry weight of sinapic acid, and/or about 210-240 μg/g dry weight of diosmin, about 4-5 μg/g dry weight of orientin, 4-6 μg/g dry weight of swertisin, and/or about 0.1-0.2 μg/g dry weight of disomentin. The extract derived from sugar cane may be in a powder form.

In one embodiment, the extract derived from sugar cane of the present disclosure comprises about 107 μg/g dry weight of syringic acid, and/or about 74 μg/g dry weight of chlorogenic acid, and/or about 7.5 μg/g dry weight of caffeic acid, and/or about 2 μg/g dry weight of vanillin, and/or about 1.7 μg/g dry weight of sinapic acid, and/or about 227 μg/g dry weight of diosmin, and/or about 4.5 μg/g dry weight of orientin, 5.2 μg/g dry weight of swertisin, and/or about 0.16 μg/g dry weight of disomentin. The extract derived from sugar cane may be in a powder form.

The extract derived from sugar cane of the present disclosure may contain a range of organic acids that are found naturally in sugar cane. These organic acids may include, but are not limited to, aconitic (cis- and trans-), oxalic, citric, lactic, tartaric, glycolic, succinic, citric, malic, fumaric and shikimic acids. In one embodiment, the extract derived from sugar cane contains higher levels of citric and malic acids than other organic acids. In another embodiment, the extract derived from sugar cane contains low to trace amounts of oxalic, citric, tartaric, glycolic, succinic and citric acids. In another embodiment, the two most abundant organic acids in the extract derived from sugar cane are trans- and cis-aconitic acids.

The extract derived from sugar cane of the present disclosure may contain trans- and/or cis-aconitic acids. In one embodiment, the extract derived from sugar cane of the present disclosure comprises trans-aconitic in amount of about 10,000 -40,000 mg per kg and/or cis-aconitic in amount of about 3,000-7,000 mg/kg. In one embodiment, the extract derived from sugar cane of the present disclosure contains trans-aconitic in an amount of about 17,000-30,000 mg per kg and/or cis-aconitic in amount of about 4,000-6,500 mg/kg. In one embodiment, the extract derived from sugar cane of the present disclosure may contain trans-aconitic in amount of about 20,000-25,000 mg per kg and/or cis-aconitic in amount of about 5,000-5,500 mg/kg.

The extract derived from sugar cane of the present disclosure may contain amino acids. In one embodiment, the total amino acids levels of the extract derived from sugar cane of the present disclosure is about 50,000-80,000 μg per gram, or about 60,000-70,000 μg per gram, or about 65,000 μg per gram. In one embodiment, about 10-40% of these total amino acids are essential amino acids. In one embodiment, about 15-30% of these total amino acids are essential amino acids. In one embodiment, about 20-25% of these total amino acids are essential amino acids.

The extract derived from sugar cane of the present disclosure may contain free amino acids. In one embodiment, the extract derived from sugar cane of the present disclosure comprises about 10,000-50,000 μg of free amino acids per gram. In one embodiment, the extract derived from sugar cane of the present disclosure may contain about 20,000-35,000 μg of free amino acids per gram. The extract derived from sugar cane of the present disclosure may contain about 25,000-30,000 μg of free amino acids per gram.

As defined above, the term “free amino acids” as used herein refers to amino acids which are singular molecules and structurally not attached to peptide bonds which are attached to other amino acids.

The extract derived from sugar cane of the present disclosure may contain leucine, a branched chain essential amino acid. In one embodiment, the concentration of leucine in the extract derived from sugar cane, is about 1-5 mM, or about 1.5-4 mM, or about 2-3 mM. In one embodiment, the amount of leucine in the extract derived from sugar cane is about 1,000-20,000 μg per gram, or about 1,000-10,000 μg per gram, or about 1,000-5,000 μg per gram, or about 1,000-2,000 μg per gram, or about 5,000-10,000 μg per gram, or about 10,000 - 20,000 μg per gram.

The extract derived from sugar cane of the present disclosure may contain minerals. In one embodiment, the extract derived from sugar cane contains minerals that are found naturally in sugar cane. In one embodiment, the extract derived from sugar cane contains one or more minerals including, but not limited to, potassium, sodium, calcium, magnesium, iron, zinc, selenium and chromium.

In one embodiment, the extract derived from sugar cane contains minerals bound to the polyphenols. In one embodiment, the extract derived from sugar cane contains divalent ions bound to the polyphenols. In one embodiment, the extract derived from sugar cane contains calcium, magnesium and/or iron bound to the polyphenols. In one embodiment, the extract derived from sugar cane contains iron bound to the polyphenols.

In one embodiment, the extract derived from sugar cane of the present disclosure comprises about 20,000-32,000 mg of potassium per kilogram, and/or about 300-600 mg of sodium per kilogram, and/or about 800-1,300 mg of calcium per kilogram, and/or about 3,000-6,000 mg of magnesium per kilogram, and/or about 40-90 mg of iron per kilogram, and/or about 3-10 mg of zinc per kilogram, and/or about 500-900 μg of selenium per kilogram and/or about 1,000-1,600 μg of chromium per kilogram. The extract derived from sugar cane may be in a syrup form.

In one embodiment, the extract derived from sugar cane of the present disclosure comprises about 25,000-27,000 mg of potassium per kilogram, and/or about 400-500 mg of sodium per kilogram, and/or about 1,000-1,200 mg of calcium per kilogram, and/or about 4,000-5,500 mg of magnesium per kilogram, and/or about 55-75 mg of iron per kilogram, and/or about 5.5-7.5 mg of zinc per kilogram, and/or about 700-850 μg of selenium per kilogram, and/or about 1,200-1,400 μg of chromium per kilogram. The extract derived from sugar cane may be in a syrup form.

In one embodiment, the extract derived from sugar cane of the present disclosure comprises about 26,000 mg of potassium per kilogram, and/or about 450 mg of sodium per kilogram, and/or about 1,090 mg of calcium per kilogram, and/or about 4,700 mg of magnesium per kilogram, and/or about 65 mg of iron per kilogram, about 6.6 mg of zinc per kilogram, and/or about 786 μg of selenium per kilogram and/or about 1,300 μg of chromium per kilogram. The extract derived from sugar cane may be in a syrup form.

In one embodiment, the extract derived from sugar cane of the present disclosure comprises about 50-350 mg of potassium per kilogram, and/or about 5-70 mg of sodium per kilogram, and/or about 7,000-10,000 mg of calcium per kilogram, and/or about 1,000-3,000 mg of magnesium per kilogram, and/or about 500-1,300 mg of iron per kilogram. The extract derived from sugar cane may be in a powder form.

In one embodiment, the extract derived from sugar cane of the present disclosure comprises about 100-250 mg of potassium per kilogram, and/or about 10-50 mg of sodium per kilogram, and/or about 8,000-9,000 mg of calcium per kilogram, and/or about 1,500-2,500 mg of magnesium per kilogram, and/or about 800-1,000 mg of iron per kilogram. The extract derived from sugar cane may be in a powder form.

In one embodiment, the extract derived from sugar cane of the present disclosure comprises about 190 mg of potassium per kilogram, and/or about 30 mg of sodium per kilogram, and/or about 8,800 mg of calcium per kilogram, and/or about 2,000 mg of magnesium per kilogram, and/or about 890 mg of iron per kilogram. The extract derived from sugar cane may be in a powder form.

The extract derived from sugar cane of the present disclosure may contain monosaccharides, disaccharides, oligosaccharides and/or polysaccharides. Examples of these include, but are not limited to, sucrose, glucose, galactose, xylose, ribose, mannose, rhamnose, fructose, maltose, lactose, maltotriose, xylopyranose, raffinose, 1-kestose, theanderose, 6-kestose, panose, neo-kestose, nystose, glucans and xylans.

In one embodiment, the compositions and methods comprises from about 0.00001 wt % to about 10 wt % of the extract.

In one embodiment, the composition and methods comprises from about 0.00001 wt % to about 50 wt % of the extract.

In one embodiment, the composition and methods comprises from about 0.00001 wt % to about 50 wt % of the extract.

In one embodiment, the composition and methods comprises from about 5 wt % to about 10 wt % of the extract.

In one embodiment, the composition and methods comprises from about 0.05 wt % to about 10 wt % of the extract.

In one embodiment, the composition and methods comprises from about 0.05 wt % to about 5 wt % of the extract.

In one embodiment, the virus is a coronavirus.

In one embodiment, the virus is an influenza virus, including H1N1, swine flu and bird flu.

In one embodiment, the virus is SARS-CoV-2.

In one embodiment, the virus is a flavivirus, including dengue, zika, west nile and yellow fever.

In one embodiment, the virus is hendra.

In one embodiment, the disease is caused by protozoa such as malaria, dysentery, giardia, cryptosporidium, Chaga's disease and coccidiosis.

In one embodiment, the agricultural greenhouse gas emissions are caused by Archael methanogens.

In one embodiment, the disease is caused by a microsporidium such as Enterocytozoon hepatopenaei.

In one embodiment, the composition for use in the inhibition and/or treatment of viral infections is a topical wash, preferably a hand wash.

In one embodiment, the composition for use in the inhibition and/or treatment of viral infections is an oral formulation, preferably a mouthwash, a gargle or liquid for ingestion or a solid such as a tablet, capsule, lozenge or confectionary. Especially preferred is a capsule.

In one embodiment, the composition for use in oral administration may be included in a liquid, solid or semi-solid foodstuff.

In one embodiment, the composition may be included in a non-human animal feed.

In one embodiment, the composition use in the inhibition and/or treatment of viral infections is an aerosol for pulmonary administration.

In one embodiment, the composition use in the inhibition and/or treatment of viral infections is an intra-nasal spray or is formulated for nasal administration.

In one embodiment, the composition use in the inhibition and/or treatment of viral infections is formulated for administration by a nebuliser.

In one embodiment, the composition use in the inhibition and/or treatment of viral infections is formulated for intravenous administration.

In one embodiment, the composition use in the inhibition and/or treatment of viral infections is formulated for pulmonary administration.

In one embodiment, the composition use in the inhibition and/or treatment of viral infections is formulated for buccal or sublingual administration.

In one embodiment, the composition use in the inhibition and/or treatment of viral infections is formulated for transdermal delivery.

The preparation of the extracts of sugar cane of this disclosure has been extensively described in WO 2019/028506. In particular page 17 to page 40 line 22 of WO 2019/028506, incorporated herein by cross reference, describe the preparation of these extracts.

Similarly in WO 2019/028506 page 50 line 16 to page 51 line 24, incorporated herein by cross reference, compositions of the extracts of sugar cane are described. Further, in WO 2019/028506 page 64 to page 88 line 9, the characteristics of the extracts of sugar cane are described.

More generally, the disclosure of WO 2019/028506 is incorporated herein by cross-reference.

A detailed analysis of a sample a sugar cane extract of this disclosure has revealed the following components:
Chlorogenic acid 2423 ng/mL
Vanillic acid 16088 ng/mL
Caffeic acid 482 ng/mL
Trans caffeic acid 273 ng/mL
p Coumaric acid 14445 ng/mL
   In addition, luteolin, trans resveratrol, diosmetin, swertisin and rutin were found to be present along with the flavonoids, apigenin-6-C-arabinosyl-8-C glucoside, apigenin-6-C-glucosyl-8-C arabinoside, apigenin-6, 8-C-diglucoside, apigenin-6″-O-glucosyl-8-C glucoside, schaftoside-2″-O-glucoside, apigenin-6-C-rhamnosyl-8-C glucoside, apigenin-6, 8-C-diarabinoside, methoxyluteolin-8-C-glucoside, methoxyluteolin-6″-O-rutinosyl-8-C-glucoside, methoxyluteolin-6″-O-glucosyl-8-C-glucoside, tricin 7-O-neohesperidoside, tricin 7-O-rhamnosyl-glucuronide and tricin 7-O-glycoside.

Formulation of the extracts into compositions of the present disclosure suitable for human use may be prepared by any of the methods well known in the art of formulation. Exemplary techniques for formulation of the compositions of the present disclosure may be found in “Remington's Pharmaceutical Sciences”, Mack Publishing Co., Easton Pa., 22^{nd} edition, 2012.

In one embodiment, an extract of this disclosure may be spray dried and directly incorporated into a capsule for human use. Typically such a composition would not require an additional carrier although carriers such as lactose may be used. For products of this type, a capsule may contain 10 to 1000 mg. Preferred is a capsule containing 100 to 500 mg of spray dried material, particularly preferred is a 250 mg capsule or 500 mg capsule..

In one embodiment, an extract of this disclosure may be spray dried and formulated into a sublingual tablet for uptake into the buccal cavity of humans. For products of this type, a tablet may contain 10 to 1000 mg. Preferred is a tablet containing 100 to 500 mg of spray dried material, particularly preferred is a 250 mg tablet or 500 mg tablet of spray dried material.

In one embodiment, an extract of this disclosure may be formulated into a liquid or semi-solid, such as a syrup or gel for sublingual uptake into the buccal cavity of humans. For products of this type, the liquid or gel may contain 10 to 30% w/w of extract. Preferred is a concentration of 15 to 20% w/w or 20 to 30% w/w.

Spray drying of extracts of this disclosure may be prepared by known methods. Such methods may or may not include a carrier such as a maltodextrin.

In one embodiment the extracts may be spray dried at 100% concentration with the spray drier inlet temperature at 140-150° C. and an outlet temperature at 90-95° C.

In one embodiment the extracts may be spray dried at 80% concentration using a maltodextrin carrier with the spray drier inlet temperature at 150-160° C. and an outlet temperature at 90-95°.

In one embodiment, an extract of this disclosure may be formulated into a spray for nasal or oral application in humans. For products of this type, the spray may contain 0.01 to 10% w/v or 0.01 to 5.0% w/v or 0.1 to 2.0% w/v or 1.0 to 2.5% w/v.

Compositions suitable for use in non-human animals have been extensively described in WO 2019/028506, for example at to and to [254], the contents of which are incorporated herein by cross-reference.

In one embodiment, in compositions suitable for use in non-human animals, an extract of this disclosure may be incorporated in feeds at a concentration of 0.001%-10% w/w or w/v for a liquid feed.

In one embodiment, in compositions suitable for use in non-human animals, an extract of this disclosure may be incorporated in feeds at a concentration of 0.01%-5% w/w or w/v for a liquid feed.

In one embodiment, in compositions suitable for use in non-human animals, an extract of this disclosure may be incorporated in feeds at a concentration of 0.1%-2% w/w or w/v for a liquid feed.

In one embodiment, in compositions suitable for use in ruminants, an extract of this disclosure may be incorporated in feeds at a concentration of 0.001%-10% w/w preferably 0.01%-5%, most preferably 0.1%-2% or % w/v for a liquid feed in order to achieve methane reductions of 3 to 80%.

In one embodiment, in compositions suitable for use in ruminants, an extract of this disclosure may be incorporated in feeds at a concentration of 0.001%-10% w/w preferably 0.01%-5%, most preferably 0.1%-2% or w/v for a liquid feed in order to achieve methane reductions of 5 to 70%.

In one embodiment, in compositions suitable for use in ruminants, an extract of this disclosure may be incorporated in feeds at a concentration of 0.001%-10% w/w preferably 0.01%-5%, most preferably 0.1%-2% or w/v for a liquid feed in order to achieve methane reductions of 10 to 60%.

In one embodiment, in compositions suitable for use in ruminants, an extract of this disclosure may be incorporated in feeds at a concentration of 0.001%-10% w/w preferably 0.01%-5%, most preferably 0.1%-2% or w/v for a liquid feed in order to achieve methane reductions of 5 to 50%.

In one embodiment, in compositions suitable for use in ruminants, an extract of this disclosure may be incorporated in feeds at a concentration of 0.001%-10% w/w preferably 0.01%-5%, most preferably 0.1%-2% or w/v for a liquid feed in order to achieve methane reductions of 5 to 40%.

In one embodiment, in compositions suitable for use in ruminants, an extract of this disclosure may be incorporated in feeds at a concentration of 0.001%-10% w/w preferably 0.01%-5%, most preferably 0.1%-2% or w/v for a liquid feed in order to achieve methane reductions of 10 to 30%.

In one embodiment, in compositions suitable for use in ruminants, an extract of this disclosure may be incorporated in feeds at a concentration of 0.001%-10% w/w preferably 0.01%-5%, most preferably 0.1%-2% or w/v for a liquid feed in order to achieve methane reductions of 5 to 10%.

In one embodiment, in compositions suitable for use in ruminants, an extract of this disclosure may be incorporated in feeds at a concentration of 0.001%-10% w/w preferably 0.01%-5%, most preferably 0.1%-2% or w/v for a liquid feed in order to achieve in order to achieve enhanced milk yields to 1 to 15% or 2 to 10% or 3 to 7%.

In one embodiment, in compositions suitable for use in non-human aquatic animals, such as shrimps and prawns, an extract of this disclosure may be incorporated in the aquatic environment in a concentration of 0.1 to 2.0% v/v.

In one embodiment, in compositions suitable for use in non-human aquatic animals, such as shrimps and prawns, an extract of this disclosure may be incorporated in the aquatic environment in a concentration of 0.1 to 1.0% v/v.

In one embodiment, in compositions suitable for use in non-human aquatic animals, such as shrimps and prawns, an extract of this disclosure may be incorporated in the aquatic environment in a concentration of 0.1 to 0.6% v/v.

In one embodiment, in compositions suitable for use in non-human aquatic animals, such as shrimps and prawns, an extract of this disclosure may be incorporated in the aquatic environment in a concentration of 0.3 to 0.6% v/v.

An example lof the preparation of the extracts of this disclosure will now be described. This extract is designated Polygain.
Vinasse is stored in tanks with recirculation prior to use.
Vinasse feedstock is passed through a 10 micron mesh to remove any large particles.
The Feedstock is then subjected to high shear mixing.
Following mixing, it is heated to 55° C.
The heated material is filtered through a 0.1 mircon membrane filtration system.
Following filtration, it is evaporated to 60 brix.
If it is to be stored as a liquid, it is heated to 80° C. and packed.
If spray dried material is required, the 60 brix product is blended with maltodextrin and mixed well.
The so-formed slurry is spray dried with inlet temperature range of 150-160° C. and outlet temperature range of 90-95°.

The polyphenol content of the Polygain was determined as follows:

| Polyphenol | Molecular formula |
|---|---|
| Chlorogenic acid | C₁₆H₁₈O₉ |
| Trans-caffeic & Caffeic acid | C₉H₈O₄ |
| Syringic acid | C₉H₁₀O₅ |
| Trans-p-coumaric | C₉H₈O₃ |
| Trans sinapic acid | C₁₁H₁₂O₅ |
| Vitexin | C₂₁H₂₀O₁₀ |
| Swertisin | C₂₂H₂₂O₁₀ |
| Homoorientin & Orientin | C₂₁H₂₀O₁₁ |
| Quercetin | C₁₅H₁₀O₇ |
| Apigenin | C₁₅H₁₀O₅ |
| Tricin | C₂₃H₂₄O₁₂ |

An example 2 of the preparation of the extracts of this disclosure will now be described. This extract is designated Virofonol.
1. Mix 300 kg of sugar cane extract with 900L of hot water (80-85° C.).
2. Mix well and check brix is 20 then add to settling tank with FPX-66 resin.
3. Mix for 20 minutes then allow the resin to settle for 15-20 minutes.
4. Pass through a 200 micron pre-filter into a tank.
5. Drain and dispose of the remaining liquid.
6. Rinse the resin with approximately 1000 L of cold water. Mix well and drain.
7. Add ethanol 71-76% to the settling tank with resin.
8. Mix for 20 minutes then allow the resin to settle for 15-20 minutes.
9. Pass through 200 micron pre-filter into a tank and collect the liquid. Repeat steps 7-9
11. Once all the liquid is collected, add 500 L of warm water (35-40°) to the resin.
12. Mix for 20 minutes then allow the product to settle for 15-20 minutes.
13. Drain and collect the liquid.
14. Add all the liquid collected into an evaporator and concentrate to 45 Brix.
15. Spray dry 45 Brix liquid with inlet temperature range of 140-150° C. and the outlet temperature range of 90-95° C. sieve and pack.

A further example of this disclosure will now be described, as Example 3.

Testing Protocol: Four solutions were evaluated to determine if any could directly inactivate SARS-CoV-2. Of these, solution 4 was a composition of the present disclosure and water was used as a negative control.

Protocol 1: 50 uL of SARS-CoV-2 stocks (undiluted) was added to 50 uL of each solution. After 1 min (simulates length of time spent in the mouth), 500 uL infection media was added to ‘quench’ the disinfection reaction. Samples were then plated directly onto TCID₅₀ plates (n=4/dilution) as soon as possible. The percentage of wells positive for CPE will be recorded, compared to the positive control sample.

100 uL of SARS-CoV-2 stock was exposed to equal volumes (100 uL) of neat and diluted solutions. After lmin, the virus:substrate solution was serially diluted and a TCID₅₀ performed. The change in the TCID₅₀/mL was compared to the positive control sample.

50% Tissue Culture Infectious Dose assay (TCID₅₀): In the PC2 laboratory, plates to establish -95% monolayers of Vero cells were seeded 24 h prior to assay. After verification of quality/density of monolayer, the plates were washed using infection media (to remove any cell debris), then transferred into the PC3 laboratory. Samples were generated, serially diluted and a known volume inoculated into each well. n=4 replicates/sample. Plates were incubated to enable virus infection of monolayers, then MEM infection media (containing pen/strep, glutamine, HEPES, but does not contain FBS)+TPCK trypsin(1 ug/mL) was added. Plates were returned to incubator (37° C., 5% CO₂) and microscopically examined every 24 h (up to 72 h) for cytopathic effect (CPE) on cells. The TCID₅₀ /mL of infectious virus present in the original sample was then determined via the method of Reed and Meunch 1938. “A simple method of estimating fifty per cent endpoints” The American Journal of Hygiene:3.

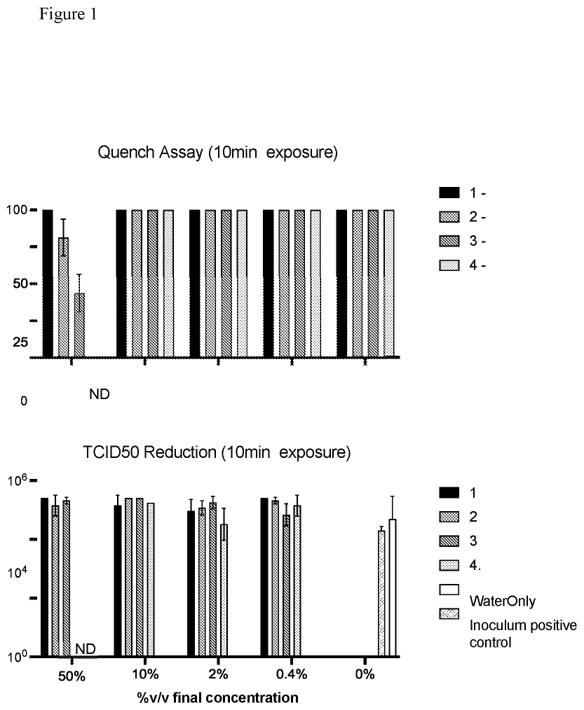

Results: when exposed directly to SARS-CoV-2, solution 4 exhibited viricidal activity that was completely effective. For both the quench and TCID50 assay, when solution 4 was used undiluted, no virus induced CPE could be detected. By contrast, no viricidal effect was evident of solutions 1 & 2. Some viricidal effect was evident for solution 3. The results are graphically shown in FIG. 1.

In a further Example 4, a composition of this disclosure was evaluated in a SARS-CoV-2 viral yield reduction assay. sARs-cov-2 patient isolate (Australia/VIC01/2020) virus was used with a sample of the composition being resuspended at 10 mg/mL in infection media (MEM+Abx+HEPES, serum free). Samples were tested at 10 ug/mL, 5 ug/mL, 2.5 ug/mL, 1.25 ug/mL and 0.625 ug/mL (final concentration after addition of virus=5, 2.5, 1.25, 0.625 and 0.313 ug/mL).

Testing Protocol: Seed Vero cells in 24-well plates one day prior to experiment start.
Infect the next day with an 100TCID50 in 100 uL and incubate for 1 h at 37° C.
New stocks were used (TCID50/mL=10^{5.59}).
Back titration yielded 943±233 TCID₅₀ in 100 uL inoculum for this experiment
Compounds were resuspended at 10 mM (from the original 10 mg/mL stocks) in DMSO, immediately prior to diluting in infection media to the dose range indicated above and used on the same day.
After 1 h incubation with virus, antiviral compounds were added in a volume of 100 μF media followed 1 hour later by addition of 0.8 ml of infection media containing 1.2 μg/ml of Trypsin (total volume in well=1 mL)
Immediately following addition of media, 125 uL was sampled for t=0 titre. 125 uL was replaced, to keep volume in well at 1 mL.

At 24 h and 48 h post infection, an aliquot of supernatant was collected and TCID50 performed to assess infectious virus titre.

50% Tissue Culture Infectious Dose assay (TCID₅₀):
In the PC2 laboratory, plates to establish ˜95% monolayers of Vero cells were seeded 24 h prior to assay
After verification of quality/density of monolayer, the plates were washed using infection media (to remove any cell debris), then transferred into the PC3 laboratory
Samples were generated, serially diluted and a known volume inoculated into each well. n=4 replicates/sample.
Plates were incubated to enable virus infection of monolayers, then MEM infection media (containing pen/strep, glutamine, HEPES, but does not contain FBS)+TPCK trypsin(1 ug/mL) was added.
Plates were returned to incubator (37° C., 5% CO₂) and microscopically examined every 24 h (up to 72 h) for cytopathic effect (CPE) on cells
The TCID₅₀/mL of infectious virus present in the original sample was then determined via the method of Reed and Meunch.

Results: Log TCID₅₀/mL values at 24 h post-infection:

| | 5 | 2.5 | 1.25 | 0.625 | 0.325 | Virus Only |
|---|---|---|---|---|---|---|
| Compound | 2.38 | 2.55 | 3.8 | 4.55 | 3.8 | 4.55 |
| concentration | 0 | 3.05 | 3.3 | 3.05 | 4.55 | 4.05 |
| (ug/mL) | 0 | 3.55 | 3.05 | 4.55 | 3.9 | 4.55 |
| | 2.8 | 3.05 | 3.05 | 3.99 | 4.05 | 3.9 |

Results: TCID₅₀/mL values at 48 h post-infection:

| Compound concentration | % |
|---|---|
| (ug/mL) | reduction |
| 5 | 99.36 |
| 2.5 | |
| 1.25 | 95.46 |
| 0.625 | 92.21 |
| 0.325 | 39.42 |

In a further example 5, vero cells were infected with West Nile virus for two hours and then treated with different concentrations of a composition of this disclosure.

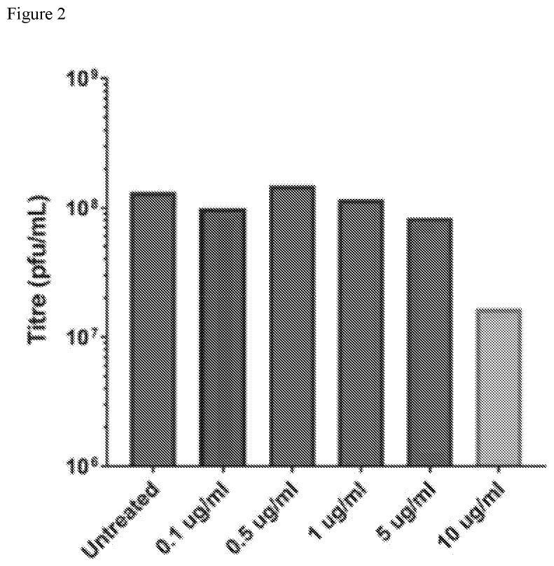

The results for example 5 are shown in FIG. 2.

In a further example 6, six species of coccidian protozoa were cultured in 19800 of RPMI medium. These were isolates of the sporozoite lifestage. These species included; Eimeria maxima, Eimeria acervulina, Eimeria brunetti, Eimeria tenella, Eimeria necatrix, Eimeria mitis. These were then treated with 20 μl of a composition of this disclosure.

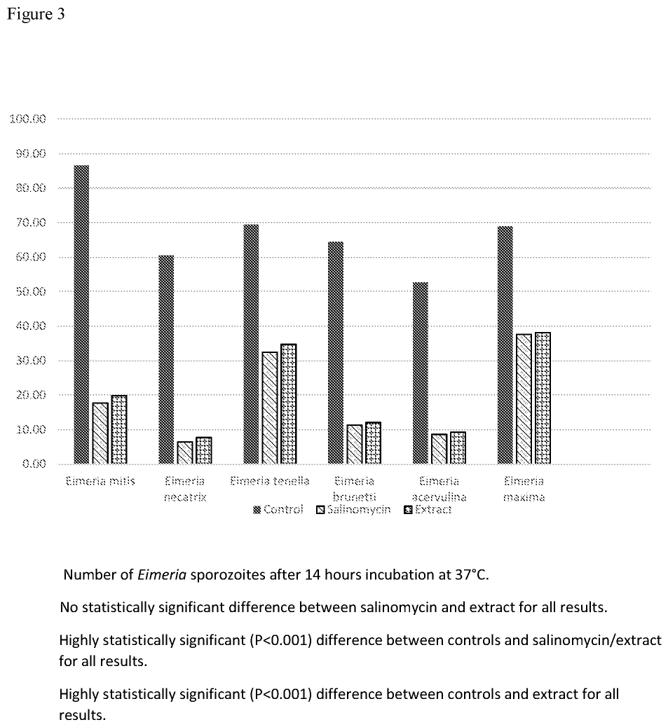

The results are shown in FIG. 3.

In a further example 7, a coccidia challenge trial was performed using Ross 308 male boiler chickens. 1 day old chickens were allocated to experimental groups that included different concentrations of a composition of this disclosure in feed, or the antibiotic ionophore salinomycin in feed or a control group containing no additive in feed. On day 14 the birds were administered a 20 times dosage of a live vaccine preparation containing 3 strains of Eimeria. These included Eimeria Maxima, Eimeria acervulina and Eimeria tenella. 7 days after the Eimeria challenge was administered birds from each treatment group were euthanized and the lesions formed in the gastrointestinal tract by the coccidia infection were scored by a qualified veterinarian.

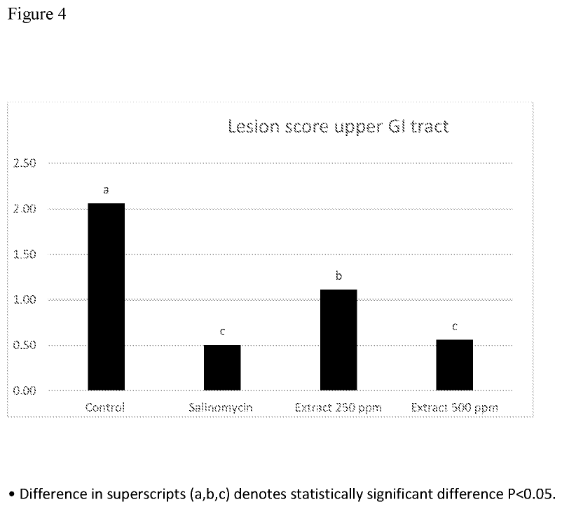

The results are shown in FIG. 4.

In a further example 8, the faecal microbiome of 8 thoroughbred horses was genetically sequenced across 5 days to determine the baseline microbial communities present. 100 ml of a composition of this disclosure was administered every 24 hours for 28 days. The 5 day genetic sequencing of faecal samples was then repeated. The administration of the extract then ceased. After a further 28 days the faecal microbiomes of the 8 horses was again repeated.

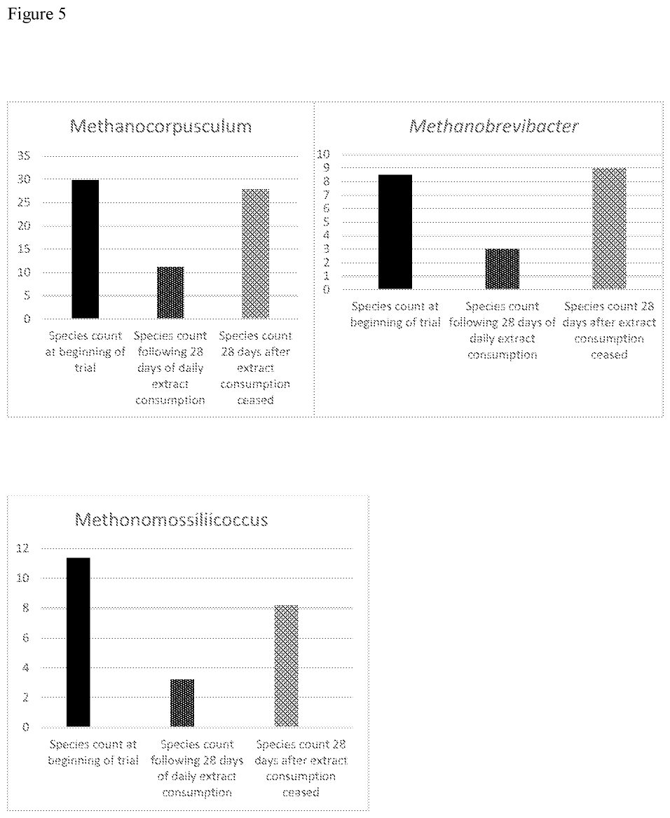

The results are shown in FIG. 5.

In a further example 9, an assessment was made of the production of infectious influenza A virus (IAV) progeny in the presence of a range of concentrations of a composition of this disclosure (referred to in this example as “Virofonol”) in-vitro (added post-exposure of cells to virus). Quantitative assays were used to measure infectious virus titre.

Influenza A Virus (IAV) (A/Beijing 89 strain—a representative seasonal strain of the H3N2 subtype) was used. Final concentrations of a composition of this disclosure (Virofonol) added after infection with virus for 1 hr—10, 5, and 2.5 m/mL. Virus recovery was assessed at 2 and 24 h post-infection (hpi). The 2 h time point was used to evaluate the residual input virus and the 24 h time point evaluates infectious virus after replication and virus production.

The infection protocol used was as follows:
Seed MDCK cells in 12-well plates one day prior to experiment start. Reserve one well to count cells before infection.
Next day, wash cells 2 times with infection media and count cells. Infect with an MOI=1.0 in 150 uL and incubate for 1 h at 37° C. Gently rock the plates every 10 minutes.
Resuspend the compound at 10 mg/mL in infection media. Filter through a 0.22 μm syringe filter.
Dilute the compound to 10, 5, and 2.5 μg/mL in infection medium.

After 1 h incubation, the inoculum is removed, and all wells are washed 2 times with infection medium.
The diluted antiviral compound is immediately added in a volume of 1 mL per well.

At 2 and 24 hours post infection (hpi), the supernatant is collected and spun down for 5 min at 13000 rpm in a microcentrifuge. (i.e.: cell free supernatants containing virus are harvested).
The clarified supernatant is stored in 2 aliquots at −80° C.
Plaque assay was performed to assess infectious virus titre.

The plaque assay protocol used was as follows:
Place 2×L15 media into the waterbath at 45° C.
Virus samples are pre-activated with trypsin Worthington at 4 ug/ml to ensure the IAV HA is cleaved.
Perform serial 10-fold dilutions of virus samples in cold serum-free RPMI+.
Aspirate media from six-well plates confluent with MDCK cells and replace with 2 ml of serum-free RPMI+. Repeat.
Aspirate RPMI+and add up 150 μl of virus dilution to duplicate wells.
The plates are then placed at 37° C./5% CO2 for 1 hour to allow the virus to infect.
Heat agarose in a microwave until agarose is completely dissolved and place in water bath at 45° C.
Add agarose overlay (3 ml in total) consisting of L15 media containing 0.9% (w/v) agarose and 2 ug/ml of trypsin Worthington.
Allow overlay to solidify before returning cells to the incubator for 3 days 37° C./5% CO₂ to allow virus plaques to form.
Count plaques by holding the plate up to the light.

The results obtained were as follows (Table 1):

TABLE 1

| | | | | | | % of reduction |
|---|---|---|---|---|---|---|
| | | | Titre (PFU/mL) | | | from virus only |
| | | Treatment | Replicate 1 | Replicate 2 | Replicate 3 | (mean) |
| 2 | hpi | virus only | 106.67 | 40.00 | 53.33 | 0.00 |
| 24 | hpi | Virofonol 2.5 μg/mL | 80.00 | 53.33 | 26.67 | 20.00 |
| | | Virofonol 5 μg/mL | 26.67 | 40.00 | 26.67 | 53.33 |
| | | Virofonol 10 μg/mL | 26.67 | 26.67 | 0.00 | 73.33 |
| | | mock-infected | 0.00 | 0.00 | 0.00 | 100.00 |
| | | virus only | 56000.00 | 49333.33 | 45333.33 | 0.00 |
| | | Virofonol 2.5 μg/mL | 38666.67 | 32000.00 | 30666.67 | 32.74 |
| | | Virofonol 5 μg/mL | 26666.67 | 26666.67 | 22666.67 | 49.56 |
| | | Virofonol 10 μg/mL | 28000.00 | 30666.67 | 21333.33 | 46.90 |
| | | mock-infected | 0.00 | 0.00 | 0.00 | 100.00 |

It will be seen that Virofonol has exhibited anti-viral effects against a representative seasonal IAV strain when added post infection, suggesting an intracellular mechanism of action. Note that no significant change was observed at 2 hpi which is an assessment of input virus before replication is established. The greatest antiviral effect was observed using concentrations of Virofonol exceeding 5 ug/mL added 1 hour post-infection and analysed 24 hours post-infection. This suggests that Virofonol can exert antiviral activity on IAV at a specific stage in the virus life-cycle after the virus has entered and initiated replication.

In a further example 10, an assessment was made of the production of infectious IAV progeny in the presence of a range of concentrations of Virofonol during and after infection in-vitro. TCID50 assay was utilised as a quantitative measure of infectious virus titre as this provides greater technical replicates using less sample and all assays can be performed within the same plate ensuring greater consistency.

Influenza A Virus (IAV) (A/Beijing 89 strain—a representative seasonal strain of the H3N2 subtype) was used. Final concentration during and after addition of virus=5, and 2.5 μg/mL. Virus recovery was at 2 and 24 h post-infection (hpi).

The infection protocol used was as follows:
Seed MDCK cells in 12-well plates one day prior to experiment start. Reserve one well to count cells before infection.
Next day, wash cells 2 times with infection media and count cells.
Resuspend the compound at 10 mg/mL in infection media. Filter through a 0.22 μmsyringe filter.
Dilute the compound to 10, 5, and 2.5 μg/mL in infection medium.
Incubate the virus for an MOI=1.0 in 150 uL/well for 1 h at 37° C. with each concentration of the diluted compound in infection medium.
Infect the cells with the compound-incubated virus for 1 h. Remove the inoculum. Wash all wells 2 times with infection medium.
Add the correspondent diluted antiviral compound again in a volume of 1 mL per well.
At 2 and 24 hpi, the supernatant is collected and spun down for 5 min at 13000 rpm in a microcentrifuge (harvested cell-free supernatant).
The clarified supernatant is stored in 2 aliquots at −80° C.
TCID50 assay was performed to assess infectious virus titre.

The TCID50 protocol used was as follows:
Seed MDCK cells in 96-well plates one day prior to the experiment. Prepare 8 wells per virus dilution. One plate per sample.
Next day, wash the cells 2 times with infection medium.
Add trypsin Worthington to infection medium at a final concentration of 2 ug/ml.
Virus samples are pre-activated with trypsin Worthington at 4 ug/ml to ensure the IAV HA is cleaved.
Dilute the virus in a 1:10 ratio in infection medium and transfer 100 ul of each dilution into 6 wells. Use column 7 as a positive control (known titre virus) and column 8 as a negative control.
Incubate at 37 in 5% CO2 for 3 days. Monitor every day.
Calculate the estimated PFU/mL through standard conversion from the TCID50.

The results obtained were as follows (Table 2):

TABLE 2

| | | | Titre (PFU/mL) determined | | | % of reduction |
|---|---|---|---|---|---|---|
| | | | from TCID50. | | | from virus only |
| | | Treatment | Replicate 1 | Replicate 2 | Replicate 3 | (mean) |
| 2 | hpi | virus only | 3533.36 | 2412.97 | 3533.36 | 0.00 |
| 24 | hpi | Virofonol 2.5 μg/mL | 3533.36 | 1643.94 | 3533.36 | 8.11 |
| | | Virofonol 5 μg/mL | 3533.36 | 1120.00 | 3533.36 | 13.64 |
| | | Virofonol 10 μg/mL | 3533.36 | 3533.36 | 1643.94 | 8.11 |
| | | mock-infected | 0.00 | 0.00 | 0.00 | 100.00 |
| | | virus only | 76123.96 | 76123.96 | 35333.61 | 0.00 |
| | | Virofonol 2.5 μg/mL | 35333.61 | 35333.61 | 3541.75 | 60.44 |
| | | Virofonol 5 μg/mL | 2412.97 | 7630.47 | 7630.47 | 90.58 |
| | | Virofonol 10 μg/mL | 7612.40 | 16400.41 | 11173.47 | 81.24 |
| | | mock-infected | 0.00 | 0.00 | 0.00 | 100.00 |

It will be seen that Virofonol has exhibited anti-viral activity against a representative seasonal IAV strain when incubated with the virus as a pre-infection and post-infection treatment. These findings suggest possible viricidal effects of Virofonol directly against the virus as well as cellular mechanisms of action by the compound. Note that no significant change was observed at 2 hpi which is an assessment of input virus before replication is established. The greatest effect was observed with concentrations of Virofonol greater than 5 μg/mL, when viral titres were quantitated 24 hpi.

In a further example 11, an assessment was made of the production of infectious ZIKV virion progeny in the presence of a range of concentrations of Virofonol added during and after infection in-vitro. TCID50 assay was utilised as a quantitative measure of infectious virus titre as this provides greater technical replicates using less sample and all assays can be performed within the same plate ensuring greater consistency. Zika virus (ZIKV) Asian was used with final concentration during and after addition of virus=10, 5, and 2.5 μg/mL. The time points were 24 and 48 h post-infection (hpi).

The infection protocol used was as follows:
Seed Vero cells in 12-well plates one day prior to experiment start. Reserve one well to count cells before infection.
Next day, wash cells 2 times with warm PBS and count cells.
Resuspend the compound at 10 mg/mL in infection media. Filter through a 0.22 μm syringe filter.
Dilute the compound to 10, 5, and 2.5 μg/mL in infection medium.
Incubate the virus for an MOI=1.0 in 300 uL/well for 1 h at 37° C. with each concentration of the diluted compound in infection medium.
Infect the cells with the compound-incubated virus for 1 h. Remove the inoculum. Wash all wells 2 times with warm PBS.
Add the correspondent diluted antiviral compound again in a volume of 1 mL per well.
At 24 and 48 hpi, the supernatant is collected and spun down for 5 min at 13000 rpm in a microcentrifuge.
The clarified supernatant is stored in 2 aliquots at −80° C.
TCID50 assay was performed to assess infectious virus titre.

The TCID50 protocol used was as follows:
Seed Vero cells in 96-well plates one day prior to the experiment. Prepare 8 wells per virus dilution. One plate per sample.
Next day, wash the cells 2 times with warm PBS.
Dilute the virus in a 1:10 ratio in infection medium (reduced serum) and transfer 100 ul of each dilution into 6 wells. Use column 7 as a positive control (known titer virus) and column 8 as a negative control.
Incubate at 37° C. in 5% CO₂ for 5 days. Monitor every day.
Calculate estimated PFU/mL from the TCID50.

The results obtained were as follows (Table 3):

TABLE 3

| | | Titre (PFU/mL) determined | | | % of reduction |
|---|---|---|---|---|---|
| | | from TCID50. | | | from virus only |
| | Treatment | Replicate 1 | Replicate 2 | Replicate 3 | (mean) |
| 24 hpi | virus only | 259928.97 | 120648.34 | 259928.97 | 0.00 |
| 48 hpi | Virofonol | 82196.76 | 25992.90 | 56000.00 | 74.37 |
| | 2.5 μg/mL | | | | |
| | Virofonol 5 μg/mL | 25992.90 | 17708.75 | 38152.36 | 87.22 |
| | Virofonol | 56000.00 | 82196.76 | 120648.34 | 59.59 |
| | 10 μg/mL | | | | |
| | mock-infected | 0.00 | 0.00 | 0.00 | 100.00 |
| | virus only | 12064834.26 | 56000000.00 | 25992897.47 | 0.00 |
| | Virofonol | 12064834.26 | 12064834.26 | 17708754.90 | 55.52 |
| | 2.5 μg/mL | | | | |
| | Virofonol 5 μg/mL | 12064834.26 | 8219675.90 | 12064834.26 | 65.61 |
| | Virofonol | 38152355.87 | 25992897.47 | 12064834.26 | 18.98 |
| | 10 μg/mL | | | | |
| | mock-infected | 0.00 | 0.00 | 0.00 | 100.00 |

Virofonol has exhibited anti-viral effects against ZIKV-Asian when incubated with the virus as a pre-infection treatment and maintained in cell culture during the assay. These findings suggest the possible extra-cellular viricidal effects of Virofonol (directly against the virus), as well as an intracellular mechanism of action by the compound (acting on cells). Note that no significant change was observed at 24 hpi which is an assessment of input virus before replication is completely established at 48 hpi. The greatest effect was observed with 5 μg/mL Virofonol after 48 hpi. This suggests that Virofonol effects the virus particle itself (acting against the virus) and/or its ability to infect and replicate within cells (cellular effects). Also note that ZIKV has a longer replication cycle than IAV, thus 2 and 24 hpi were assessed for IAV compared with 24 and 48 hpi for ZIKV.

Overall, examples 9-11 demonstrate that Virofonol has shown antiviral activity when applied directly to the virus inoculum itself and after the infection process for both IAV and ZIKV. These results indicate that Virofonol is useful both as an extracellular and intracellular antiviral agent.

In a further example 12, two compositions of this disclosure (referred to as Polynol and Polygain) were assessed for dose response against (infectious laryngotracheitis virus) ILTV CSW1.

In this example, Chicken (leghorn) liver hepatoma cell line (LMH cells) were seeded into a 12 well plate until 90% confluent.
Cells were infected with 400 ul of 1×10⁶ pfu/mL ILTV CSW1 virus for 1 hour.
After 1-hour, various concentrations (Polynol: 0.5, 1 and 1.5 mg/ml, Polygain: 1, 1.5 and 2 mg/ml) were added to corresponding wells.
Incubated for 48 hours. Post incubation, cell media was collected for plaque assay

The results for this example were as follows (Table 4):

TABLE 4

| Concentration (mg/ml) | % Reduction |
|---|---|
| Polynol: 0.5 | 57.77 |
| Polynol: 1 | 74.76 |
| Polynol: 1.5 | 61.62 |
| Polygain: 1 | 52.47 |
| Polygain: 1.5 | 71.05 |
| Polygain: 2 | 36.91 |

In a further example 13, a composition of this disclosure (Polygain) was evaluated for its ability to reduce methane emissions by dry cows.

Dry cows were fed 3 kg concentrate mix (control) or concentrate mixed with 100 g Polygain. Measurements of methane concentration were determined at 11 am and again at 1 pm.

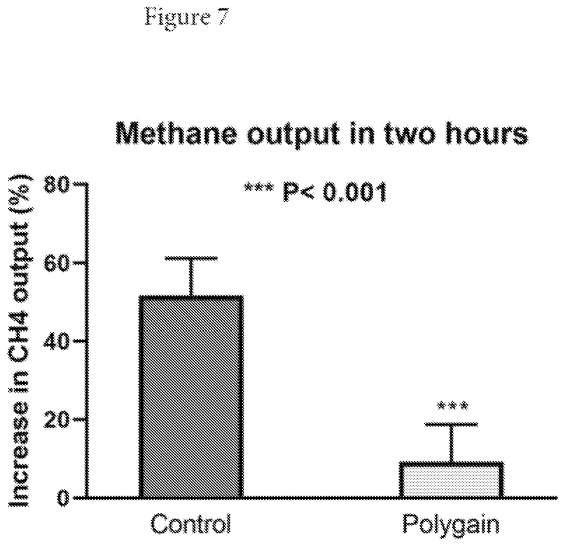

The results in FIG. 7 clearly show that Polygain reduces methane emission significantly during the first two hours when compared to the control group. These results demonstrate the dysbiosis effect of the compositions of this disclosure.

In a further example 14 a composition of this disclosure (Polygain) was evaluated for its ability to reduce methane generation in vitro. In this example, a sample of rumen fluid was obtained and used to ferment samples of a typical feedlot diet or Rhodes grass. In the sample under evaluation, a concentration of 15% of Polygain was included in each of the fermentations.

The results as set out below show that for the feedlot diet, a 54% reduction in methane generation was achieved after 24 hours per gram of digested dry matter. For the Rhode grass diet, the reduction was 16.5% after 24 hours per gram of digested dry matter.

| | |
|---|---|
| Feedlot | Rhode grass |

| Parameter | 0% | 15% | 0% | 15% |
|---|---|---|---|---|
| Cumulative CH₄ 24 h, mL | 4.44 | 3.73 | 4.14 | 5.07 |
| Cumulative CH₄ 24 h, mL/g | 10.90 | 5.06 | 7.88 | 6.58 |
| digested DM | | | | |

In a further example 15, a composition of this disclosure (Polygain) was evaluated for its ability to inhibit the polar tube extrusion activity of EHP.

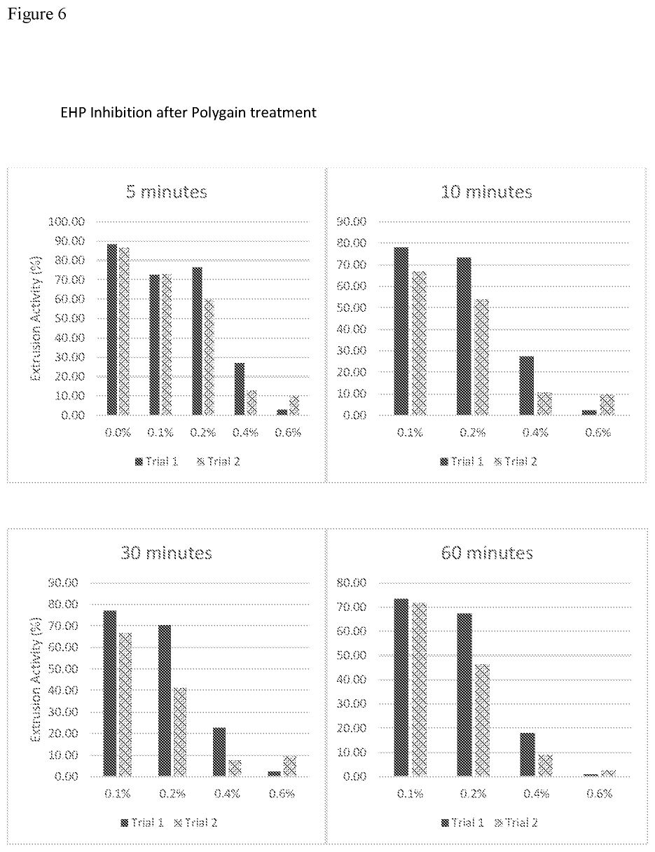

The results shown in FIG. 6 clearly illustrate that Polygain was highly effective in its inhibitory activity against EHP. Note that PBS was the control. Therefore it is evident that Polygain would function to prevent infection of prawns by EHP and indeed other aquatic species susceptible to infection by EHP.

In a further example 16, a composition of this disclosure (Polygain) was evaluated for its ability to enhance milk production in dairy cattle. In this example, cattle were either fed their normal diet (control) or their normal diet including either 0.5% or 1.0% Polygain. Milk volume and concentrate were measured on a daily basis with the results shown in Table 5.

TABLE 5

| | Polygain concentration | Milk | Concentrate/100 |
|---|---|---|---|
| Day | (% w/w) | (litres/day/cow) | kg milk |
| 7 | 0 | 33.2 | 23.9 |
| 7 | 0.5 | 34.9 | 22.9 |
| 7 | 1.0 | 35.3 | 22.7 |
| 41 | 0 | 31.0 | 25.4 |
| 41 | 0.5 | 32.2 | 25.7 |
| 41 | 1.0 | 32.8 | 24.6 |

These results show that at 7 days, daily milk production was increased by 2.1 L/day/cow in the cows that were supplemented with Polygain.. This was continued at 41 days where milk production was increased by 1.8 l/day/cow in the cows that were supplemented with Polygain.. These indicate increases of 6.3% and 5.8% respectively.

In addition, protein and fat content were maintained for all of the cows that were supplemented with Polygain.

Finally, a lower level of concentrate was required in the Polygain supplemented cows in order to achieve an enhanced milk yield.

In a further example 17, a composition of this disclosure (Polygain) was evaluated for its ability to enhance the growth of calves. In this trial, calves were either fed their normal diet (control) or their normal diet including 10 g Polygain.

The diet consisted of 4.0 kg wheat, 1.0 kg corn, 1.0 kg canola meal, 0.1 kg limestone, 0.1 kg salt, 0.05 kg dicalcium phosphate, 0.04 kg magnesium oxide and vitamin and trace mineral premix. It was fed as a pellet or loose grain mix.

Results of the trial are set out in Table 6. These show that the average total weight gain for the control group was 67.6 kg as compared with 70.5 kg for the test diet including Polygain. Average daily weight gain for the control group was 0.82 kg compared with 0.86 kg. This amounts to a difference of 4.9% which is highly significant in terms of the economic value of each animal.

Taken in conjunction with the example relating to reduction in methane production, it is evident that the disclosed sugar extracts provide a double benefit. Body mass is enhanced whilst methane production is reduced.

TABLE 6

| Trial | Weight of Test Subjects - Control Group [kg] | | | | | | | | | |
|---|---|---|---|---|---|---|---|---|---|---|
| date | 1 | 2 | 3 | 4 | 5 | 6 | 7 | 8 | 9 | 10 |
| 4-Aug | 45.0 | 48.0 | 43.0 | 33.5 | 44.0 | 38.5 | 40.0 | 44.0 | 48.5 | 44.0 |
| 4-Sep | 68.0 | 73.0 | 65.0 | 56.0 | 59.5 | 49.5 | 54.0 | 61.0 | 65.0 | 62.5 |
| 25-Oct | 123.5 | 138 | 109 | 98 | 111 | 89 | 105.5 | 104.5 | 123.5 | 113 |
| Weight gain | 55.5 | 65.0 | 44.0 | 42.0 | 51.5 | 39.5 | 51.5 | 43.5 | 58.5 | 50.5 |
| between 25 Oct | | | | | | | | | | |
| and 4 Sep | | | | | | | | | | |
| Total weight | 78.5 | 90.0 | 66.0 | 64.5 | 67.0 | 50.5 | 65.5 | 60.5 | 75.0 | 69.0 |
| gain | | | | | | | | | | |

| Trial | Weight of Test Subjects - Control Group [kg] | | | | | | |
|---|---|---|---|---|---|---|---|
| date | 11 | 12 | 13 | 14 | 15 | 16 | 17 |
| 4-Aug | 46.5 | 43.0 | 49.0 | 37.0 | 41.5 | 42.0 | 46.5 |
| 4-Sep | 65.5 | 63.5 | 66.0 | 52.5 | 53.0 | 58.0 | 66.0 |
| 25-Oct | 114 | 104.5 | 118.5 | 94.5 | 109.5 | 101.5 | 125 |
| Weight gain | 48.5 | 41.0 | 52.5 | 42.0 | 56.5 | 43.5 | 59.0 |
| between 25 Oct | | | | | | | |
| and 4 Sep | | | | | | | |
| Total weight | 67.5 | 61.5 | 69.5 | 57.5 | 68.0 | 59.5 | 78.5 |
| gain | | | | | | | |

| | Test Subjects - Control Group | | |
|---|---|---|---|
| | Average | Average | Average |
| Trial | weight of | weight | weight gain |
| date | 1-17 [kg] | gain [kg] | per day [kg] |
| 4-Aug | 43.2 | — | — |
| 4-Sep | 61.1 | 17.9 | — |
| 25 Oct | 110.7 | 49.7 | 0.97 |
| Average weight | 49.7 | | |
| gain between | | | |
| 25 Oct and 4 Sep | | | |
| Average total | 67.6 | | 0.82 |
| weight gain | | | |

| Trial | Weight of Test Subjects - Polygain feed [kg] | | | | | | | | |
|---|---|---|---|---|---|---|---|---|---|
| date | 1 | 2 | 3 | 4 | 5 | 6 | 7 | 8 | 9 |
| 4-Aug | 45.0 | 48.0 | 42.0 | 37.0 | 43.5 | 38.5 | 43.0 | 48.0 | 43.0 |
| 4-Sep | 63.0 | 67.0 | 65.5 | 57.0 | 64.5 | 54.5 | 63.0 | 67.5 | 58.0 |
| 25-Oct | 115 | 120 | 121.5 | 113 | 132.5 | 101.5 | 127.5 | 126.5 | 106.5 |
| Weight gain | 52.0 | 53.0 | 56.0 | 56.0 | 68.0 | 47.0 | 64.5 | 59.0 | 48.5 |
| between 25 Oct | | | | | | | | | |
| and 4 Sep | | | | | | | | | |
| Total weight | 70.0 | 72.0 | 79.5 | 76.0 | 89.0 | 63.0 | 84.5 | 78.5 | 63.5 |
| gain | | | | | | | | | |

| Trial | Weight of Test Subjects - Polygain feed [kg] | | | | | | |
|---|---|---|---|---|---|---|---|
| date | 10 | 11 | 12 | 13 | 14 | 15 | 16 |
| 4-Aug | 47.0 | 43.5 | 46.0 | 37.0 | 41.5 | 35.5 | 48.0 |
| 4-Sep | 69.0 | 58.5 | 58.0 | 51.5 | 57.5 | 54.5 | 62.5 |
| 25-Oct | 113.5 | 106 | 102 | 103.5 | 107.5 | 104 | 114.5 |
| Weight gain | 44.5 | 47.5 | 44.0 | 52.0 | 50.0 | 49.5 | 52.0 |
| between 25 Oct | | | | | | | |
| and 4 Sep | | | | | | | |
| Total weight | 66.5 | 62.5 | 56.0 | 66.5 | 66.0 | 68.5 | 66.5 |
| gain | | | | | | | |

| | Test Subjects - Polygain feed | | |
|---|---|---|---|
| | Average | Average | Average |
| Trial | weight of | weight | weight gain |
| date | 1-16 [kg] | gain [kg] | per day [kg] |
| 4-Aug | 42.9 | — | — |
| 4-Sep | 60.7 | 17.8 | — |
| 25 Oct | 114.5 | 52.7 | 1.03 |
| Average weight | 52.7 | | |
| gain between | | | |
| 25 Oct and 4 Sep | | | |
| Average total | 70.5 | | 0.86 |
| weight gain | | | |

In a further example 18, a composition of this disclosure (Polygain) was evaluated for its ability to improve the health of dairy cattle by preventing and/or treating mastitis. Conductivity data was collected from over 4000 milking sessions from 27 cows over a four month period broken up into a control group and a group having 1% w/w Polygain, on a total dry matter basis, included in their diet.

As previously explained, milk conductivity is directly indicative of the presence of mastitis in a cow. Generally, conductivity increases with incidence of mastitis. It is to be noted that it is unusual for the four quarters of a cow (teats) to be all infected. Therefore by comparing the lowest conductivity quarter with each other quarter as a ratio is indicative of mastitis being present in one or more quarters. Such measurements are typically collected automatically in so-called robotic milking installations. Of course, milk samples may be manually acquired and the conductivity evaluated on a quarter by quarter basis.

In this example 18, the samples were acquired in a robotic milking installation. As shown in Table 7, not only did the average base conductivity decrease over time but the average ratio between the quarters did as well. This is strongly indicative of improved cow mammary gland health likely due to prevention, treatment and/or reduction in the incidence of mastitis.

TABLE 7

| Dependent Variable | Treatment | Mean |
|---|---|---|
| Total Conductivity | Control | 74.524 |
| Baseline | Polygain | 70.989 |
| Inter-quarter ratio | Control | 1.088 |
| Conductivity Baseline | Polygain | 1.042 |
| Conductivity End | Control | 75.206 |
| | Polygain | 70.963 |
| Inter-quarter ratio | Control | 1.104 |
| Conductivity Post | Polygain | 1.037 |
| Total Conductivity | Control | 0.682 |
| difference over trial | Polygain | −0.026 |
| Inter-quartile ratio | Control | 0.016 |
| difference over trial | Polygain | −0.006 |

In a further example 19, a composition of this disclosure (Polygain) was evaluated for its ability to reduce methane emissions by dry cows. Polygain was included in the feed for each of the test groups of cows at 0.5% or 1% w/w on a dry matter basis.

At the commencement of the trial, methane concentrations were captured at periodic times over about a 42 hour period. Methane concentrations were again captured after 32 days of feeding at periodic times over about a 42 hour period.

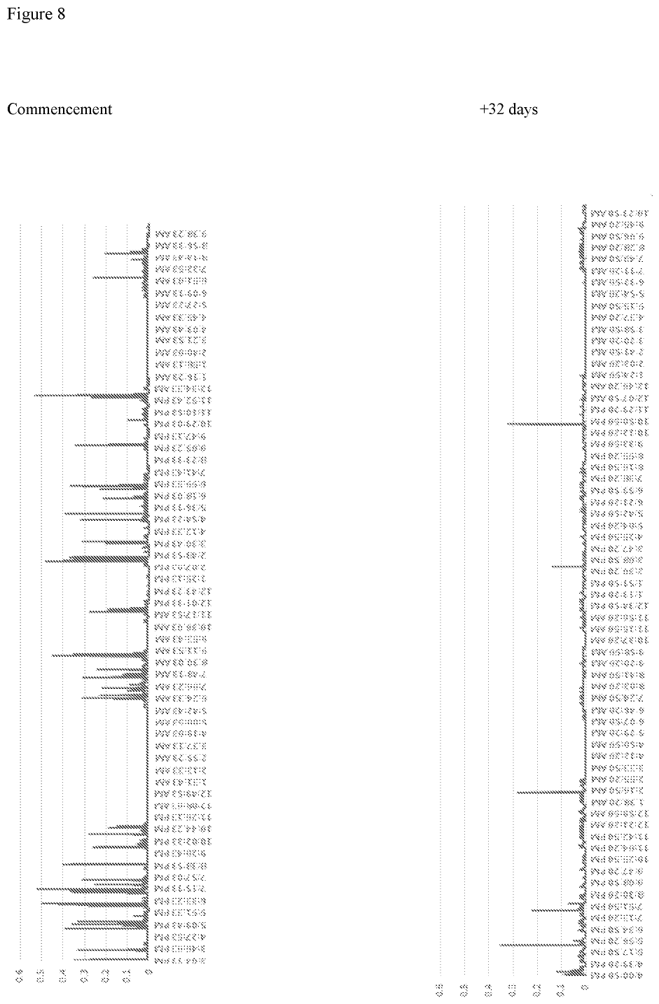

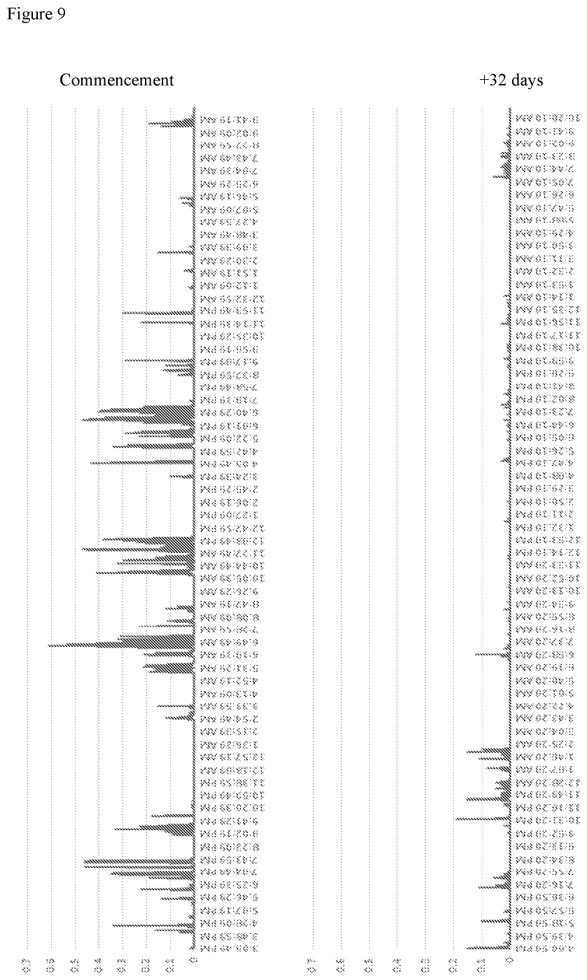

The results of the trial at 0.5% Polygain are shown in FIG. 8 and the results for 1% Polygain are shown in FIG. 9. Referring to FIG. 8, qualitatively it can be seen that the methane concentration in the 0.5% test group recorded four values only above 0.2. By comparison at commencement, numerous values in excess of 0.2 were recorded.

Referring to FIG. 9, qualitatively it can be seen that the methane concentration in the 1% test group did not exceed 0.2. By comparison at commencement, numerous values in excess of 0.2 were recorded.

Thus this example 19 shows clearly the dysbiosis effect compositions of this disclosure.

In a further example 20, the faeces of a cow that had been fed on a control diet and a second cow that had been fed on the same diet with the addition of 100 g Polygain were evaluated for methanogenic archea populations.

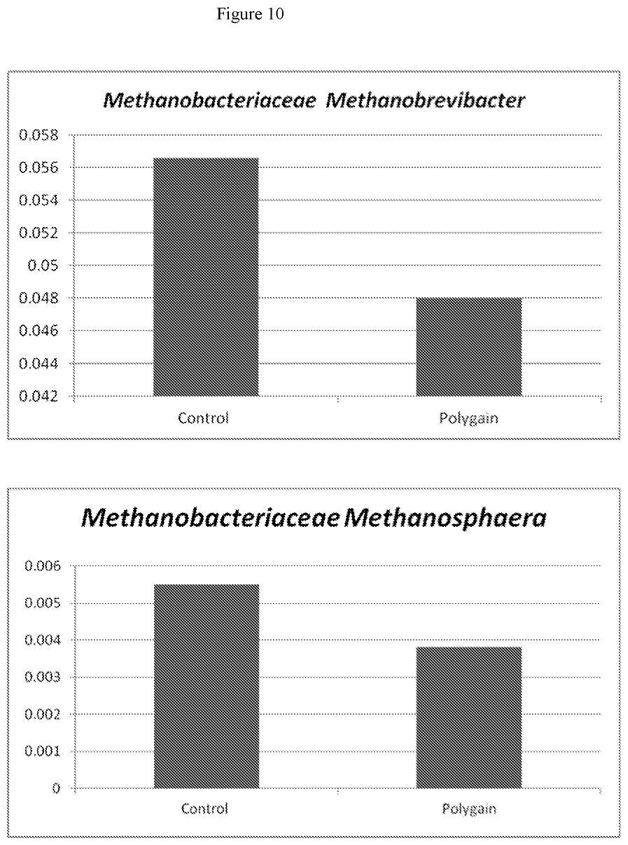

The results of example 20 are depicted in FIG. 10. These show that for Methanobacteriaceae Methanobrevibacter, the reduction in population of this microorganism was about 15% whilst for Methanobacteriaceae Methanosphaera, the reduction was about 30%. Such results reinforce the findings in other examples disclosed here that the methane reducing effect of Polygain is due to changes induced in the microbiome of ruminants. Specifically, the populations of methane generating microorganisms.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A composition comprising from about 0.00001 wt % to about 100 wt % of an extract derived from sugar cane for use in the inhibition or inactivation of viruses or the prevention or treatment of viral infections in a subject, the extract comprising from about 10 catechin equivalent (CE) g/L to about 50 CE g/L of polyphenols or from about 100 CE mg/g to about 500 CE mg/g of polyphenols, and wherein the use is by a topical, a pulmonary, a respiratory, an intravenous or an oral route.

2. A method of inhibiting or inactivating viruses or the prevention or treatment of viral infections in a subject comprising administering to the subject by a topical, a pulmonary, a respiratory, an intravenous or an oral route an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

3. The composition of claim 1 or the method of claim 2 wherein the composition comprises from about 0.05 wt % to about 10 wt % of the extract.

4. The composition or method of claim 3 wherein the composition comprises from about 0.05 wt % to about 5 wt % of the extract. The composition or method of any one of claims 1 to 4 wherein the virus is a coronavirus, an influenza virus or a flavivirus.

6. The composition or method of claim 5 wherein the virus is SARS-CoV2, H1N1, swine flu, bird flu, zika, dengue, yellow fever, hendra or west nile.

7. The composition or method of any one of claims 1 to 6 wherein the composition is a topical wash, preferably a hand wash.

8. The composition or method of any one of claims 1 to 6 wherein the composition is an oral formulation, preferably a mouthwash, a gargle, a liquid for ingestion, a capsule, tablet, lozenge or confectionary product.

9. The composition or method of claim 8 wherein the composition is a capsule.

10. The composition or method of any one of claims 1 to 6 wherein the composition is an intra-nasal spray.

11. A composition comprising from about 0.00001 wt % to about 100 wt % of an extract derived from sugar cane for use in the prevention or in the treatment of an aquatic animal by inhibition or inactivation of microsporidian parasites, the extract comprising from about 10 catechin equivalent (CE) g/L to about 50 CE g/L of polyphenols or from about 100 CE mg/g to about 500 CE mg/g of polyphenols, and wherein the use is by inclusion of the extract in an aquatic environment.

12. A method of inhibiting or inactivating microsporidian parasites in an aquatic animal comprising administering to the animal via its aquatic environment an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

13. A composition comprising from about 0.00001 wt % to about 100 wt % of an extract derived from sugar cane for use in the treatment of dysbiosis in an animal by inhibition or inactivation of methanogens, the extract comprising from about 10 catechin equivalent (CE) g/L to about 50 CE g/L of polyphenols or from about 100 CE mg/g to about 500 CE mg/g of polyphenols.

14. A method of treating dysbiosis in an animal comprising administering to the animal an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

15. A method of inhibiting or inactivating the lifecycle of protozoan parasites both in vertebrates and invertebrate animals or in an environment they inhabit, comprising administering to the animal, an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

16. A composition comprising from about 0.00001 wt % to about 100 wt % of an extract derived from sugar cane for use in inhibiting or inactivating the lifecycle of protozoan parasites both in vertebrates and invertebrate animals or in an environment they inhabit, the extract comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

17. A method of modulating the microbiome of animals, comprising administering to the animal, an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

18. A composition comprising from about 0.00001 wt % to about 100 wt % of an extract derived from sugar cane for use in modulating the microbiome of animals, the extract comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

19. A method of mitigating greenhouse gas emissions by an animal by changing the composition of microbial communities associated with the animal comprising administering to the animal, an effective amount of a composition comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

20. A composition comprising from about 0.00001 wt % to about 100 wt % of an extract derived from sugar cane for use in mitigating greenhouse gas emissions by an animal by changing the composition of microbial communities associated with the animal, the extract comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

21. A method or a composition according to claim 19 or claim 20 wherein the animal is a ruminant and the microbial communities are in the rumen.

22. A method for the prevention or treatment of mastitis in a ruminant animal comprising administering to the animal, an effective amount of a composition comprising from about 0.00001 wt % to about 100 wt % of an extract derived from sugar cane, the extract comprising from about 10 catechin equivalent (CE) g/L to about CE g/L of polyphenols or from about 100 CE mg/g to about 500 CE mg/g of polyphenols.

23. A composition comprising from about 0.00001 wt % to about 100 wt % of an extract derived from sugar cane for use in the prevention or treatment of mastitis in a ruminant animal, the extract comprising from about 0.00001 wt % to about 100 wt % of an extract derived from sugar cane, the extract comprising from about 10 catechin equivalent (CE) g/L to about 50 CE g/L of polyphenols or from about 100 CE mg/g to about 500 CE mg/g of polyphenols.

24. A composition comprising from about 0.00001 wt % to about 50 wt % of an extract derived from sugar cane for use as an adjuvant either alone or in combination with a second adjuvant, the extract comprising from about 10 catechin equivalent (CE) g/L to about 50 CE g/L of polyphenols or from about 100 CE mg/g to about 500 CE mg/g of polyphenols.

25. A method of improving the immune response of a vaccine comprising including an effective amount of a composition comprising from about 0.00001 wt % to about 50 wt % of an extract derived from sugar cane in a vaccine composition, the extract comprising from about 10 catechin equivalent (CE) g/L to about 50 CE g/L of polyphenols or from about 100 CE mg/g to about 500 CE mg/g of polyphenols.
